(19) United States
(12) Patent Application Publication
(10) Pub. No.: US 2013/0303869 A1
(43) Pub. Date: Nov. 14, 2013

(54) MOBILE ANALYTE MONITORING SYSTEM

(71) Applicant: WELLSENSE INC., Portland, OR (US)

(72) Inventors: Mihailo V. Rebec, Bristol, IN (US); Slavko N. Rebec, Bristol, IN (US); Richard G. Sass, Portland, OR (US)

(73) Assignee: WellSense Inc., Portland, OR (US)

(21) Appl. No.: 13/892,151

(22) Filed: May 10, 2013

Related U.S. Application Data
(60) Provisional application No. 61/645,929, filed on May 11, 2012.

(51) Int. Cl. A61B 5/145

(52) U.S. Cl. CPC A61B 5/14532
USPC 600/365

(57) ABSTRACT

A mobile analyte monitoring system may include an implantable sensor and a reader device with an optical sensor. The implantable sensor may be implanted into the dermis of an animal, and may exhibit a color change in response to the presence of a target analyte or reaction product thereof. The reader device may be configured to capture an image of the implanted sensor and to determine the concentration of the target analyte based at least in part on the image. One or more portions of the implantable sensor or components thereof may be configured to facilitate calibration of the sensor, correction of an optical signal obtained from the sensor by a reader device to accommodate variations in the surrounding tissues, and/or calculation of a representative value by a reader device. The reader device may be a personal electronic device such as a cell phone, PDA, or personal computer.

### TECHNICAL FIELD

Embodiments herein relate to the field of medical devices and systems, and, more specifically, to devices and systems for mobile analyte monitoring.

### BACKGROUND

Continuous long-term monitoring of medical conditions such as diabetes presents challenges for both patients and medical care providers. Traditional methods that require the patient to repeatedly obtain and test blood or other fluids can be painful and inconvenient, and this may lead to reduced compliance on the part of the patient. Implantable sensors developed to mitigate these drawbacks have been expensive, bulky, require a power source or specialized reader, or lack the necessary mechanical strength to remain functional within the patient for extended periods of time. In addition, such sensors may be difficult to remove several weeks after implantation.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIGS. 1 a-e illustrate plan views of an implantable analyte sensor;

FIGS. 2a-c and 2d-e illustrate side views of an implantable analyte sensor as shown in FIGS. 1a and 1d, respectively;

FIG. 3 illustrates an example of a reagent system for glucose detection in an implantable sensor;

FIGS. 4a-f illustrate examples of an analyte monitoring system;

FIG. 5 illustrates an example of a logic flow diagram for an analyte monitoring system;

FIG. 6 illustrates another example of a logic flow diagram for an analyte monitoring system; and

FIGS. 7a-u illustrate examples of user interface displays corresponding to various operations of an analyte monitoring system, all in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “NB” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Embodiments herein provide methods, systems, and apparatuses for mobile monitoring of one or more analytes in an animal, such as a human. A mobile monitoring system may include an analyte sensor and a reader device. In some examples, the analyte sensor may be an implantable analyte sensor. Implantable sensors as described herein may be more robust, more easily optically read, thinner, less expensive to produce, and/or more easily removed than prior known implantable sensors. The implantable sensor may be read by a reader device such as the sensor user's existing personal device, such as a cell phone, smart phone, tablet computing device, personal digital assistant, or laptop. This further reduces the expense and increases the convenience of the mobile analyte monitoring system.

For the purposes of this description, an “implantable sensor” is a sensor that is implanted into the skin with the main body of the sensor, or a portion thereof, residing in the dermis of the skin. In some embodiments, the entirety of the implanted sensor may reside in the dermis. In other embodiments, a portion of the implanted sensor may protrude into the epidermis, extending through the outer surface or to just below the surface of the skin. The sensor or a portion thereof may be implanted to a depth of 20 μm to 1000 μm below the surface of the skin. The implantable sensor may reside in the skin for a period of time that can range from one hour to a couple of years depending upon one or more factors, such as the type(s) of analysis needed and the stability of the analysis components. The implantable sensor may be inserted and/or removed with an insertion/removal device.

In one embodiment, an implantable sensor may have a base, a body defining one or more chambers, and one or more permeability/blocking members. The base may be constructed from one or more materials such as a polymer or a metal. The body may be coupled to a surface of the base. The chambers may be one or more gaps, wells, or voids extending partially or fully through the thickness of the body. An analyte reagent system with one or more sensor reagents may be retained within a chamber. The analyte reagent system may include one or more sensor reagents configured to detect the target analyte(s). One or more permeability/blocking members may be coupled to the chambers and/or to the body. Some or all of the sensor reagents may be retained on or between the permeability/blocking member(s), or between the permeability/blocking member(s) and the body.

The analyte reagent system may be configured to respond to the presence of an analyte by changing color and/or emitting light (luminescence). In some embodiments, the analyte reagent system may be configured to respond to the presence of an analyte by a reduction in emitted light in a portion of the sensor. A sensor may include one or more analysis regions, each configured to exhibit a color or emission of light in the presence of a corresponding analyte. Some sensors may include a group or array of analysis regions configured to detect a corresponding group of analytes. In some embodiments, a sensor may include two or more analysis regions configured to detect a target analyte within different concentration ranges (e.g., one detects the analyte within a “high” concentration range and another detects the same analyte within a “low” concentration range). Some or all of the analysis regions may have different detection ranges (i.e., configured to detect analytes within different concentration ranges) but to exhibit responses within a common range of response. Thus, two analysis regions may be configured to exhibit a particular color in response to different analytes or different concentrations of the same analyte. Similarly, two analysis regions may be configured to exhibit different colors in response to a particular concentration of a particular analyte.

The sensor may include one or more control regions configured to provide a reference color, current, shape, or other parameter for use by the reader device. A control region may be an analysis region that is configured to detect a target analyte (e.g., a duplicate analysis region) or to detect a non-target analyte. Other control regions may be control elements located on or within the sensor. Control elements may be, but are not limited to, a fixed color and/or shape that can be used by the reader device as a reference. Such control regions may be provided to confirm sensor integrity, for calibration of the reader device based on implantation depth or dermal characteristics, for detection of leakages or malfunction, to orient a captured image for analysis, to assess implantation depth or sensor integrity, to determine optical corrections for differences in ambient light or light intensity, skin pigmentation/color, skin scattering, or image exposure/collection times, and/or to correct a representative value or other calculated value based on differences in the depth of the sensor in the skin (e.g., for a sensor that is placed at a greater or lesser depth in the skin than recommended).

The response or color of each analysis/control region may be read by a reader device such as mobile electronic device (e.g., a wireless phone or computer) that includes an optical sensor (e.g., a camera). The reader device may capture an image of the implanted sensor. The reader device may then determine the concentrations of one or more of the target analytes based on the captured image. The reader device may determine one or more representative values, such as a blood glucose value, that represents the determined concentration. The image, image data, or representative value(s) may be communicated by the reader device to the user, a caretaker, a medical care provider, a medical device manufacturer, a health management system, a satellite health/device management system, and/or a medical device.

Systems, methods, and apparatuses disclosed herein may allow patients, caretakers, device manufacturers, health management systems, and/or medical service providers to monitor the health of the sensor wearer more closely and conveniently. In addition, embodiments disclosed herein may allow medical device manufacturers to monitor the quality of the data or information delivered to a patient, caretaker, or medical service provider, to monitor and track sensor performance, to create and update performance logs for sensors, to change or update an algorithm of the reader device based on sensor performance (e.g., to compensate for changes in sensor responses as a result of sensor aging or deterioration), to determine or predict a recommended time or date for sensor removal or replacement, and/or to communicate relevant data regarding sensor performance to a user, caretaker, medical services provider, health management system, or other entity or system. Closer monitoring and efficient adjustment of analyte concentrations may significantly improve the quality and duration of a user's life. Sensors as described herein may be configured to monitor the concentration of a target analyte within the dermis of a user for 30, 60, 90, 180, or more than 180 days (e.g., 1 year, 1.5 years, or 2 years).

### Examples of Implantable Sensors

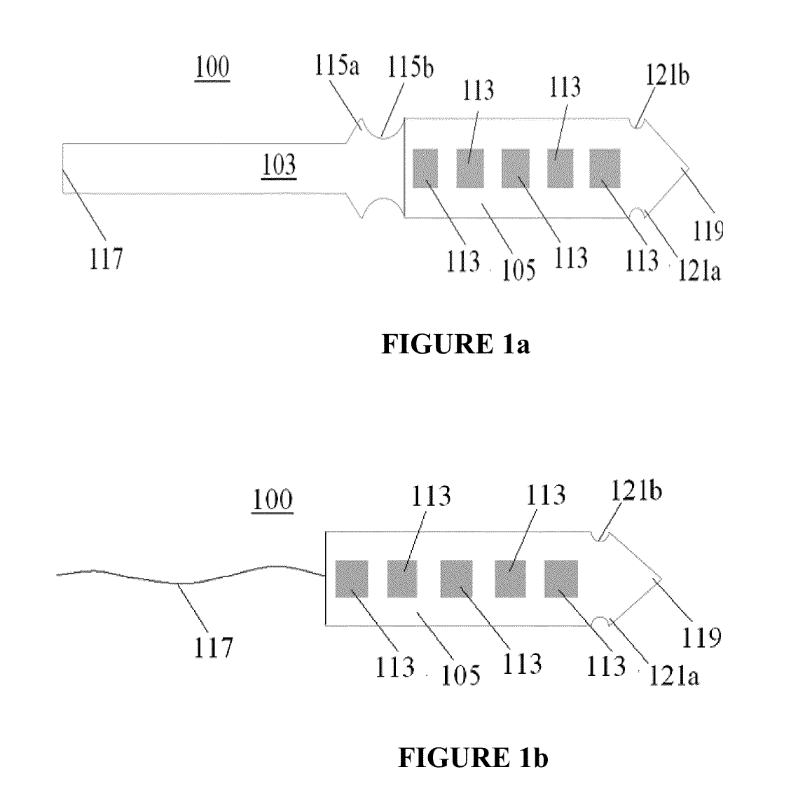

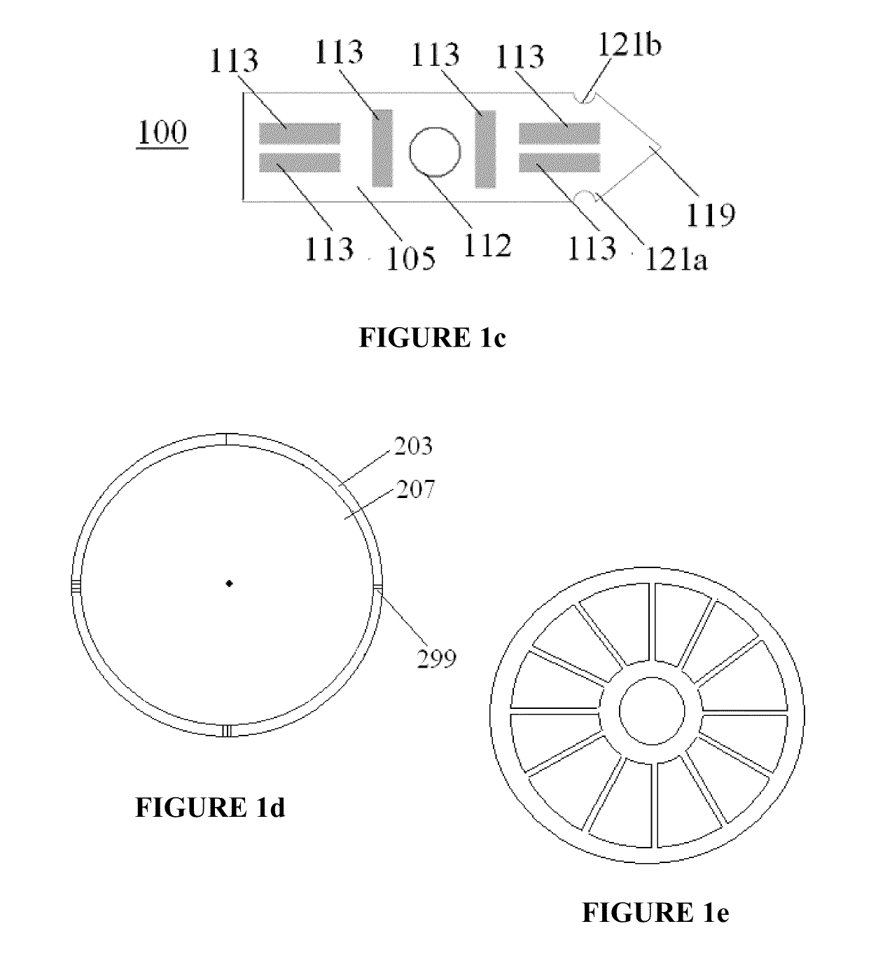

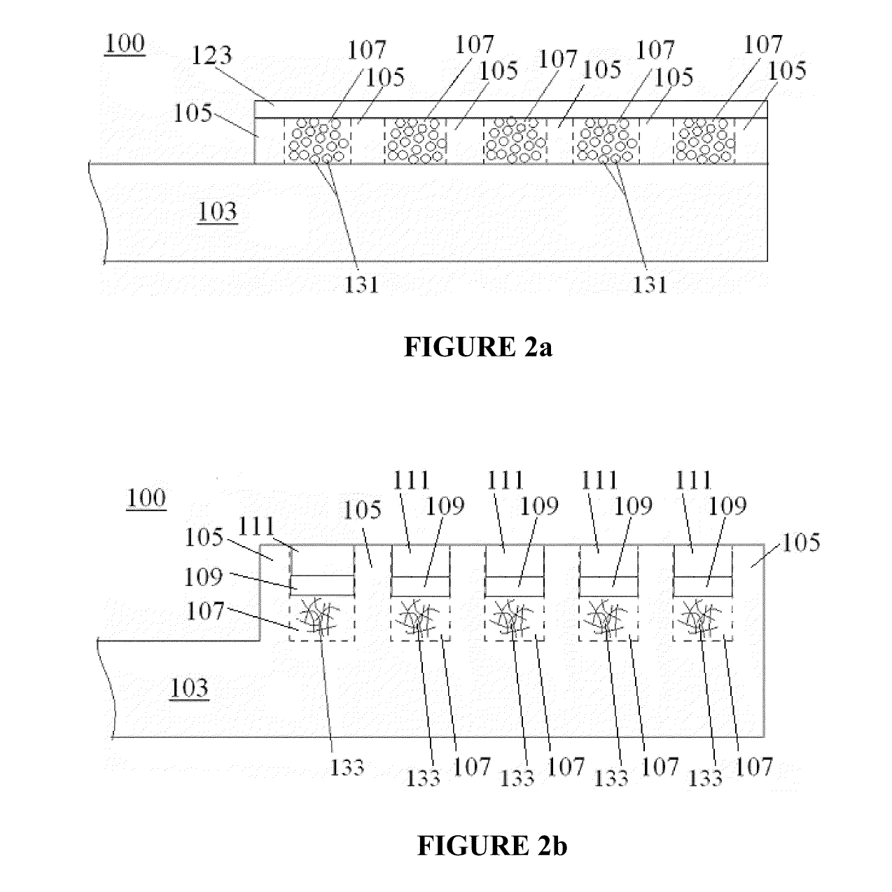

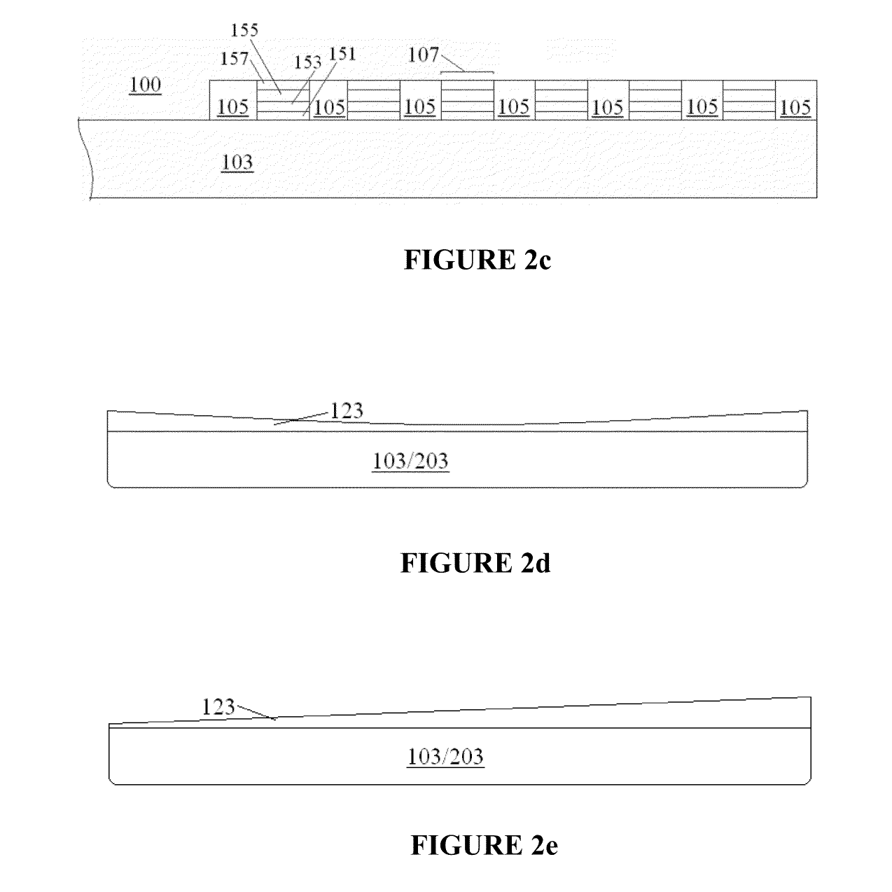

FIGS. 1a-e illustrate plan views of implantable sensors in accordance with various embodiments. FIGS. 2a-c and 2d-e illustrate side views of implantable sensors as shown in FIGS. 1a and 1d, respectively, in accordance with various embodiments.

As illustrated, an implantable sensor 100 may have a base 103 coupled to a body 105. Analysis regions 113 may be arranged along base 103 and surrounded by body 105. An analysis region may include a chamber and the analyte reagent system within the chamber. Optionally, the analysis region may also include the underlying base and/or one or more permeability/blocking member(s). Thus, a first chamber may be part of a first analysis region, a second chamber may be part of a corresponding second analysis region, and a third chamber may be part of a corresponding third analysis region. Alternatively, a chamber may represent more than one analysis region. For example, a sensor may have a single continuous chamber with a configuration that varies from one side of the chamber to another. Variations in configuration may include, for example, diffusion gradients, different concentrations of reagents, different base thicknesses, or different optical properties across the base.

Implantable sensors may have any number and combination of analysis regions configured to detect one or more analytes. Some implantable sensors may lack one or more of these analysis regions. Others may include additional analysis regions configured to detect other analytes that are relevant to the health of the animal. Optionally, an implantable sensor may include one or more additional analysis/control regions configured to serve as a control for calibration and/or to confirm correct positioning, functionality, and/or accessibility of implantable sensor 100 to the target analyte(s) or control analyte(s).

Base 103 and body 105 may form first and second layers, respectively, of implantable sensor 100 (see FIG. 2a). Alternatively, body 105 and base 103 may be formed as integral portions of a single unit (see FIG. 2b). For example, body 105 and base 103 may be a single piece formed by molding, thermoforming, vacuum forming, compaction and sintering, cutting, or extrusion of a base material. Base 103 may have an elongate shape with a first end 117 and an opposite second end 119. Second end 119 may terminate in a point or other shape to aid penetration into the skin during implantation or subsequent removal of the sensor from the skin. Base 103 may include one or more surface or edge features configured to enhance the retention of implantable sensor 100 within the dermis after implantation. In the examples of FIG. 1a, implantable sensor 100 includes projections 115a and 121a near a first end and a second opposite end, respectively, of body 105. Invaginations 115b and 121b are positioned between the projections and body 105. These features may provide resistance to backward-directed pulling forces to prevent the dislocation of the implantable sensor after implantation.

In some embodiments, second end 119 may be inserted into the dermis of an animal and first end 117 may be retained externally, above the epidermis, for removal. For example, the terminal edge (e.g., 0.5 mm) of first end 117 may protrude from the surface of the skin. In other embodiments, first end 117 may be positioned within the epidermis a short distance below the outer surface of the skin, and may become exposed for removal 1, 2, 3, 4, 5, or 6 months after implantation. In still other embodiments, first end 117 may be positioned below the epidermis after implantation. First end 117 may alternatively be positioned within the epidermis and may become exposed by natural exfoliation of the epidermis over a period of weeks or months. As another alternative, first end 117 may be inserted into the dermis of an animal and second end 119 may be retained externally (above the epidermis), within the epidermis, or below the epidermis as described above.

As shown in FIG. 1 b, first end 117 may be a relatively thin and flexible member, such as a narrow tape or string, which can be grasped and pulled to remove the sensor from the skin. Other sensors may lack an elongated end. Optionally, sensors may have a surface feature configured to mate with a portion of a removal device for removal of the sensor. For example, as shown in FIG. 1 c, a sensor may be provided with a hole 112 through a portion of the base and/or body. A portion of an insertion/removal device may be inserted through the hole and pulled to remove the sensor from the skin. The sensor may be configured to at least partially fold or collapse for removal. Some sensors may have a pointed or narrow end to aid in removal of the sensor from the dermis.

Base 103 can include one or more materials such as a metal and/or metal alloy (e.g., stainless steel), a hydrogel, a plastic or polymer, a biopolymer (e.g., a polyanhydride), ceramic, and/or silicon. Examples of plastics or polymers may include, but are not limited to, polyacrylic acid (PAA), cross-linked polyethylene (PEX, XLPE), polyethylene (PE), polyethylene terephthalate (PET, PETE), polyphenyl ether (PPE), polyvinyl chloride (PVC), polyvinylidene chloride (PVDC), polylactic acid (PLA), polypropylene (PP), polybutylene (PB), polybutylene terephthalate (PBT), polyamide (PA), polyimide (PI), polycarbonate (PC), polytetrafluoroethylene (PTFE), polystyrene (PS), polyurethane (PU), polyester (PEs), acrylonitrile butadiene styrene (ABS), poly(methyl methacrylate) (PMMA), polyoxymethylene (POM), polysulfone (PES), styrene-acrylonitrile (SAN), ethylene vinyl acetate (EVA), and styrene maleic anhydride (SMA). In one embodiment, base 103 may be magnetic. For example, base 103 may comprise 50-90% iron. In some embodiments, base 103 may comprise magnetic/magnetized stainless steel. In some examples the stainless steel can be a stainless steel of either the martensitic type or the ferritic type. Base 103 may have a thickness in the range of 30 μm to 500 μm. For example, base 103 may have a thickness in the range of 30-35 μm, 35-40 μm, 40-50 μm, 50-60 μm, 60-70 μm, 70-80 μm, 80-100 μm, 100-150 μm, 150-200 μm, 200-250 μm, 250-300 μm, 300-350 μm, 350-400 μm, 400-450 μm, or 450-500 μm.

In some sensors, ambient light may be reflected by reagents within chambers 107, and the resulting diffuse reflection signal may be measured by a reader device. Optionally, base 103 may include a reflective material that is integral (i.e., integrated within the material used to form base 103) or provided in the form of a coating along one or more surfaces of base 103, such as a coating along the bottom surface. The inclusion of reflective materials in or on base 103 may reduce background effects from tissue below the sensor and/or enhance the reflection or transflection of light from by the sensor. At least some ambient light may pass through the reagents within chambers 107 to be reflected by the reflective material of base 103. The resulting transflectance signal may be measured by a reader device. In such examples, the sensor may provide diffuse reflection signals and/or transflectance signals, and the reader may measure the signals of one or both types. In one example, base 103 includes a strip of polyimide material impregnated with titanium dioxide (TiO₂). Optionally, base 103 may be thicker at a first end than at a second, opposite end, to provide an optical gradient.

Body 105 may be constructed from a variety of materials depending on the strength and permeability desired. In some examples, body 105 may be a plastic or a polymer (e.g., polyimide). Body 105 may range in thickness from 5 μm to 500 μm thick. For example, body 105 may have a thickness in the range of 5-10 μm, 10-15 μm, 15-20 μm, 20-25 μm, 25-30 μm, 30-35 μm, 35-40 μm, 40-45 μm, 45-50 μm, 50-60 μm, 60-70 μm, 70-80 μm, 80-100 μm, 100-150 μm, 150-200 μm, 200-250 μm, 250-300 μm, 300-350 μm, 350-400 μm, 400-450 μm, or 450-500 μm. In one example, base 103 is a strip of polyimide material impregnated with TiO₂, and body 105 is polyurethane.

Body 105 can be applied onto base 103 as a liquid solution or vapor by printing, roll-coating, dip-coating, spin coating, spraying, chemical/physical vapor deposition, sol-gel, or other known methods. In some examples, the solution or vapor may be applied indiscriminately to an area of base 103. A pattern mask or other physical/chemical blocking agent may be used to prevent deposition of the solution or vapor over the areas where chambers 107 are desired. In other examples, the solution may be applied selectively to some areas of base 103, leaving other areas (e.g., chambers 107 and/or first end 117) untreated. Alternatively, body 105 may be a pre-formed solid, semi-solid, or gel, and may be coupled to base 103 with an adhesive. In some embodiments, body 105 and base 103 are formed as a single unit. Base 103 and/or body 105 can have varying thicknesses.

As best viewed in FIGS. 2a-c, one or more chambers 107 may extend partially or entirely through the thickness of body 105. Chambers 107 may be cut from body 105 before or after body 105 is applied or coupled to base 103. Alternatively, body 105 and base 103 may be a single unit, and chambers 107 may be made during formation of the unit (e.g., as part of a molding process) or after formation of the unit (e.g., by cutting or otherwise removing material from the unit).

The number, shape, depth, and spatial arrangement of chambers 107 may vary among embodiments. Similarly, the shape and depth of chambers 107 may vary within an individual sensor, with some chambers having a greater depth or different shape than others. An implantable sensor may have 2, 3, 4, 5, 6, 7, 8, 9, 10, or more than 10 chambers 107. In one example (FIG. 1a), the implantable sensor has six rectangular areas (i.e., chambers 107) that may be, for example, 300×400 μm in size. In other embodiments, one or more of chambers 107 may be round, oblong, polygonal, and/or have one or more tapered sides.

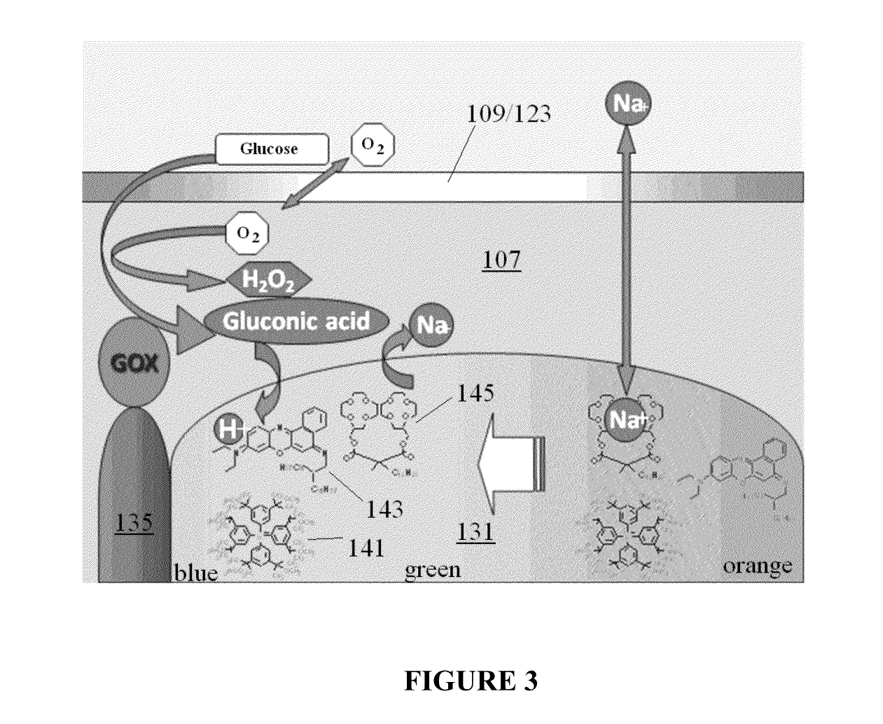

At least some of chambers 107 may contain an analyte reagent system with one or more sensor reagents, discussed further below with reference to FIG. 3. Sensor reagents may be bound to microscopic beads, fibers, membranes, gels, or other matrices in various combinations. Some sensor reagents may be retained between membranes, bound to membrane materials coated onto a membrane, or coupled/immobilized to a hydrophilic matrix. The analyte reagent system may be provided in a single layer or in multiple layers. For example, an analyte reagent system may include two, three, four, five, six, seven, eight, nine, ten, or more than ten layers.

At least one of the layers may be a permeability/blocking member, such as a membrane or gel that is selectively permeable to one or more sensor reagents, analytes, or reaction products. A permeability/blocking member may include one or more membranes and/or gels, alone or in combination. Examples of permeability/blocking members are described in U.S. Pat. No. 7,964,390, which is hereby incorporated by reference in its entirety. Permeability/blocking members may include one or more membranes, such as cellulose acetate membranes, cellulose acetate phosphate membranes, cellulose acetate pthalate membranes, and/or polyurethane membranes. Some permeability/blocking members may include, for example, a hydrogel, polyurethane, polyvinylpyrrolidone, acrylic polyesters, vinyl resins, fluorocarbons, silicones, rubbers, chitosan, hydroxyethylmethacrylate (HEMA), polyethylene glycol methacrylate (PEGMA), and/or polyhydroxyethylmethacrylate.

One or more of the layers may comprise a liquid or gel. In some embodiments, the liquid (or a liquid component of the gel) may be provided by the surrounding tissue after implantation of the sensor. For example, a layer may include one or more gel components in a dehydrated form, such as a powder, that is configured to form a gel upon exposure to tissue fluids.

FIG. 2c illustrates an embodiment of a sensor with a multi-layer analyte reagent system. In this embodiment, the analyte reagent system includes a first layer 151, a second layer 153, and a third layer 157.

First layer 151 may include a matrix and an indicator. The matrix may include one or more of a liquid, a gel, beads, fibers, a membrane or membrane component(s), and/or another porous material. Some of the sensor reagents may be dispersed in the matrix or bound to a component thereof. The indicator may be a group of sensor reagents configured to collectively provide a response, such as a color change, upon exposure to a target analyte.

An indicator may be a pH sensitive dye that produces a color change in response to a change in pH resulting from a target analyte or reaction product/intermediate. The indicator may return to its previous color when the pH returns to its previous level. An indicator may include a group of chemical species that function as a system. For example, an indicator may include one or more of an ionophore, a lipophilic anion, and a chromoionophore (i.e., a lipophilic hydrogen ion sensitive dye). The ionophore may extract the ion to be detected (e.g., hydrogen), causing the chromoionophore to change color. Electrical neutrality may be maintained by the negatively charged anion. For example, as illustrated in FIG. 3, an indicator may include a chromogen, an ionophore, and a lipophilic anion. In other embodiments, an indicator may be a luminescent reagent that emits light in response to a target analyte or reaction product/intermediate. Luminescent reagents may include, but are not limited to, photoluminescent (e.g., phosphorescent or fluorescent), chemiluminescent, electroluminescent, electrochemiluminescent, or bioluminescent reagents. Alternatively, an indicator may be an enzyme or reaction product thereof. Some embodiments may include two or more indicators in the same or different analysis regions.

In some examples, the matrix may be a membrane and the first group of sensor reagents may be immobilized on the membrane. In other examples, at least some of the sensor reagents of the indicator may be bound to a matrix component, such as beads 131 (FIG. 2a) or elements 133 (e.g., fibers, a membrane, a membrane component, or other porous material; FIG. 2b). Different sensor reagents may be bound to separate membranes, beads, or other matrix components, or to different portions of a single membrane, bead, or matrix component.

Second layer 153 may be coupled to first layer 151. Second layer 153 may include a detection reagent. A detection reagent is a reagent that reacts with, or catalyzes a reaction of, the target analyte to produce a reaction product or intermediate. A detection reagent may be an enzyme or an enzyme system. For example, a detection reagent for glucose detection may be glucose oxidase (“GOX”), and a detection reagent for lactose detection may be lactase. In some embodiments, a detection reagent may be or include an antibody that binds to an analyte or reaction product, and/or an enzyme attached to such an antibody. The binding of the antibody to the analyte or reaction product may cause a change in the activity of the enzyme, which may influence or cause a change in pH. Thus, an analyte reagent system can include any antibody, enzyme, antibody-enzyme complex, or indicator known in the art for use in the detection of analytes in vitro or in vivo.

Second layer 153 may include a liquid, a gel, beads, fibers, a membrane or membrane component(s), and/or another porous material. In some examples, second layer 153 may include a membrane that is selectively permeable to a target analyte. The membrane may be impermeable to one or more sensor reagents (e.g., detection/indicator reagents). A detection reagent may be immobilized on a membrane, beads, or other element of second layer 153.

Third layer 157 may be a permeability/blocking member that is configured to selectively limit the passage of a target analyte or interfering compounds into second layer 153.

Optionally, a fourth layer 155 may be applied to reduce or prevent damage to another layer during manufacturing. For example, fourth layer 155 may be a protective layer applied over first layer 151, and second layer 153 may be applied over fourth (protective) layer 155. This may protect first layer 151 from being damaged as second layer 153 is being applied. In some examples, fourth layer 155 may also be a permeability/blocking member such as a membrane or gel. Optionally, fourth layer 155 and/or an additional layer may be applied over some or all of the analyte sensor to enhance biocompatibility, structural intergrity, or both. For example, one or more of the outer surfaces of the analyte sensor may be coated with a layer of a biocompatible material. In some embodiments, the biocompatible material may include one or more of nafion, phosphorylcholine, polyurethane with phospholipids, and/or a hydrogel. In some embodiments, the biocompatible material may be applied to the analyte sensor by dip coating or vapor coating.

In other embodiments, some or all of the detection reagent(s) and indicator(s) may be provided within a single layer (see e.g., FIGS. 2a, 2b, and 3). The indicator and detection reagent may be immobilized within the layer on beads, membranes, fibers, or other elements. A permeability/blocking member 109 may be coupled to the chambers 107 and/or to the body 105, and the detection reagent and indicator may be retained between the permeability/blocking member 109 and the body 105. In some examples, the detection reagent and/or indicator may be bound to the underside of the permeability/blocking member 109. Permeability/blocking member 109 may include one, two, or more two layers of membrane and/or gel. Optionally, a second permeability/blocking member 111 may be added over first permeability/blocking member 109.

Permeability/blocking members of varying configurations may be used among chambers 107 to provide increased or decreased permeability to the target analyte(s) among neighboring chambers 107. For example, a first permeability/blocking member 109 of a first chamber 107 may be more or less permeable to a target analyte than a permeability/blocking member 109 of a second chamber 107. One or more of the permeability/blocking members may be configured for a desired permeability to a control analyte, such as sodium or cholesterol. Permeability/blocking members may be applied individually to chambers 107 as separate units. Alternatively, permeability/blocking member 123 may be coupled to multiple chambers 107 as a single unit, as shown in FIG. 2a.

In some embodiments, individual permeability/blocking members 109 may be coupled to corresponding chambers 107, and a single permeability/blocking member 123 may be applied as a single layer across the upper surface of body 105 (see FIG. 2b). Permeability/blocking member 123 may have different configurations at different locations along its length, such as differences in pore size(s), thickness, or other parameters. This may provide one or more chambers with different permeabilities to a target analyte or reagent (see e.g., FIGS. 2d-e).

One or more of the permeability/blocking members and chambers may be made of a set of materials with a composition that varies in permeability from one portion to another. For example, a permeability/blocking member and/or chamber can have a decrease in permeability from the upper surface to a lower portion, such that larger molecules can permeate the upper part with limited or no entry into the lower portion, but smaller molecules such as sodium and hydrogen ions can permeate the lower portion. This could be accomplished by changing the relative amounts of the polymers, cross-linking agents, and/or photoinitiators that are used or deposited in the formation of the component. Alternatively, a permeability gradient may be accomplished by provided a permeability/blocking member that is thinner at the center than at the outer edge, or thinner at one side than at another side.

Some sensors may have a single continuous chamber. For example, FIG. 1d illustrates a sensor with a round body 203, a single chamber 207, and control elements 299. Control elements such as control elements 299 may have any suitable shape, size, color, or location, and may be provided on any component or portion of an implantable sensor (e.g., to a base/body, chamber, permeability/blocking member, and/or any other component). Examples of possible control elements include a fixed color and/or shape.

Other sensors may have multiple chambers and/or analysis regions in various arrangements, such as wedges (see e.g., FIG. 1 e), rings, or other patterns. For example, a round sensor may have two or more analysis regions arranged in concentric rings. The inner ring may be configured to exhibit a response to an analyte concentration that is within a first range, and the second ring may be configured to exhibit a response to an analyte concentration that is within a second range. Alternatively, one or both of the rings may be configured for use as a control region and/or for detecting a non-target analyte.

### Examples of Analyte Reagent Systems and Components

As discussed above, an analyte reagent system may include an indicator that provides a color change and/or a spectral change in response to a target analyte. In some embodiments, the analyte reagent system may be configured to exhibit a color/spectral change in response to a corresponding change in the concentration of an analyte. An indicator may be, but is not limited to, a pH-sensitive dye with one or more chromoionophores, lipophilic anions, and/or ionophores. In other embodiments, an indicator may be, or may include, a chromoionophore that is configured to bind one or more other ions (e.g., sodium, potassium, calcium, or chloride) and to exhibit a color/spectral change in response to binding and/or release of the ion(s). Other indicators may include luminescent reagents, enzymes, and/or reaction products. For example, in some embodiments, an indicator may be a luminescent reagent that emits light in response to a target analyte or reaction product/intermediate. Luminescent reagents may include, but are not limited to, photoluminescent (e.g., phosphorescent or fluorescent), chemiluminescent, electroluminescent, electrochemiluminescent, or bioluminescent reagents. Alternatively, an indicator may be an enzyme or reaction product thereof. Some embodiments may include two or more indicators in the same or different analysis regions.

Examples of chromoionophores include, but are not limited to: chromoionophore I (9-(diethylamino)-5-(octadecanoylimino)-5H-benzo[a]phenoxazine) designated ETH5249; chromoionophore II (9-dimethylamino-5-[4-(16-butyl-2,14-dioxo-3,15 ioxaeicosyl)phenylimino]benzo[a]phenoxazine) designated ETH2439; chromionophore III (9-(diethylamino)-5-[(2-octyldecyl)imino]benzo[a]phenoxazine), designated ETH 5350; chromoionophore IV (5-octadecanoyloxy-2-(4-nitrophenylazo)phenol), designated ETH2412; chromoionophore V (9-(diethylamino)-5-(2-naphthoylimino)-5H-benzo[a]phenoxazine); chromoionophore VI (4′,5′-dibromofluorescein octadecyl ester) designated ETH7075; chromoionophore XI (fluorescein octadecyl ester) designated ETH7061; and combinations thereof.

Examples of lipophilic anions include, but are not limited to: KTpCIPB (potassium tetrakis(4-chlorophenyl)borate), NaHFPB (sodium tetrakis[3,5-bis(1,1,3,3,3-hexafluoro-2-methoxy-2-propyl)phenyl]borate), sodium tetrakis[3,5-bis(trifluoromethyl)phenyl]borate, sodium tetrakis(4-fluorophenyl)borate, combinations thereof, and the like.

Examples of ionophores include, but are not limited to: Sodium ionophores, such as bis[(12-crown-4)methyl]2-dodecyl-2-methylmalonate, designated ETH227; N,N′,N″-triheptyl-N,N′,N″-trimethyl-4,4′,4″-propylidynetris(3-oxabutyramide), designated ETH157; N,Ni-dibenzyl-N,Ni-diphenyl-1,2-phenylenedioxydiacetamide, designated ETH2120; N,N,N′,N′-tetracyclohexyl-1,2-phenylenedioxydiacetamide, designated ETH4120; 4-octadecanoyloxymethyl-N,N,N′,N′-tetracyclohexyl-1,2-phenylenedioxydiacetamide), designated DD-16-C-5; 2, 3:11,12-didecalino-16-crown-5), bis(benzo-15-crown-5), and combinations thereof; Potassium ionophores, such as: bis[(benzo-15-crown-5)-4′-methyl]pimelate, designated BME 44; 2-dodecyl-2-methyl-1,3-propanedil bis[N-{5′-nitro(benzo-15-crown-5)-4′-yl]carbamate], designated ETH1001; and combinations thereof; Calcium ionophores, such as: (−)-(R,R)—N,N′-bis-[11-(ethoxycarbonyl)undecyl]-N,N′-4,5-tetramethyl-3,6-dioxaoctane-diamide), designated ETH129; N,N,N′,N′-tetracyclohexyl-3-oxapentanediamide, designated ETH5234; N,N-dicyclohexyl-N′,N′-dioctadecyl-3-oxapentanediamide), designated K23E1; 10,19-bis[(octadecylcarbamoyl)methoxyacetyl]-1,4,7,13,16-pentaoxa-10,19-diazacycloheneicosane), and combinations thereof.

FIG. 3 illustrates an example of a reagent system with a pH-sensitive indicator for use in an implantable sensor. This reagent system provides a GOx/pH based reaction that produces a color shift (i.e., a variation in reflected wavelengths of light) that can be measured to determine a glucose concentration. In this example, the chromoionophore is chromionophore III, the ionophore is bis[(12-crown-4)methyl]2-dodecyl-2-methylmalonate, and the lipophilic anion is sodium tetrakis[3,5-bis(1,1,1,3,3,3-hexafluoro-2-methoxy-2-propyl)phenyl]borate trihydrate. In this system, the chromoionophore exhibits a pH-dependent color between the extremes of orange and blue. The pH shifts in response to varying concentrations of glucose. The reflected wavelengths (orange, yellow, green, blue) from the analysis regions can be detected and analyzed to determine the local glucose concentration.

As illustrated, glucose and oxygen enter chamber 107 through permeability/blocking membrane (109/123). Chamber 107 may include an indicator coupled to a substrate 131. In the illustrated example, the indicator includes a chromoionophore 143, an ionophore 145, and a lipophilic anion 141. A detection reagent (e.g., GOx) may be immobilized on a substrate 135. Each of substrates 131 and 135 may be an independent component such as a bead, a membrane, a fiber, or a surface of body 105 that is exposed within chamber 107. In other examples, a substrate 131 and a substrate 135 may integrated within one component.

The GOx converts glucose and oxygen to gluconic acid and hydrogen peroxide. Increasing production of gluconic acid causes a shift in pH. The chromoionophore 143 accepts a hydrogen ion, which causes a shift in the color of the chromoionophore 143 toward blue. As electrical neutrality is maintained by the lipophilic anion 141, the ionophore 145 responds to the acceptance of the hydrogen ion by releasing a sodium ion to maintain the charge balance. As the production of gluconic acid decreases, the ionophore accepts a sodium ion, and the chromoionophore releases a hydrogen ion, causing a shift in color of the chromoionophore toward orange. The shift in color causes a corresponding shift in wavelengths reflected by the analysis regions, which can be detected to monitor glucose levels at desired time intervals.

Optionally, one or more additional reagents may be provided within chamber 107. The additional reagent(s) may be provided to increase the rate of a chemical reaction, stabilize one or more components of the analyte reagent system, and/or convert a reaction product to another product. For example, catalase may be provided to convert hydrogen peroxide to water and oxygen.

In some embodiments, sensor reagents of an analyte system may be segregated within chamber 107. This may be useful where two or more sensor reagents are incompatible or require different pH levels for optimal enzyme activity or stability. For example, within chamber 107, one or more pH sensing areas with an indicator may be segregated from one or more enzyme areas with detection reagents. The sensor reagents may be deposited separately in the respective areas, such as in one or more gels or on separate substrates. The respective areas may be in direct contact. Alternatively, another substrate or material may provide a transition zone between the areas. For example, a detection reagent such as GOx may be deposited in a first (enzyme) area and an indicator may be deposited in a second (pH sensing) area. Hydrogen ions generated in the reaction area would diffuse to the pH sensing area. Optionally, the hydrogen ions may diffuse through a hydrogel disposed between the two areas.

While some implantable sensors may have two or more separate areas as described above, other sensors may have a plurality of similar but smaller micro-areas dispersed throughout a chamber 107 or along a permeability/blocking member in one or more patterns. Examples of suitable patterns include, but are not limited to, alternating dots/squares/lines and concentric circles. In a specific example, two respective areas are arranged to form two or more separate, concentric circular portions, with one of the areas (e.g., an enzyme area) disposed in an outer ring and surrounding the other area (e.g., a pH-sensing area).

### Examples of Mobile Analyte Monitoring Systems and Reader Devices

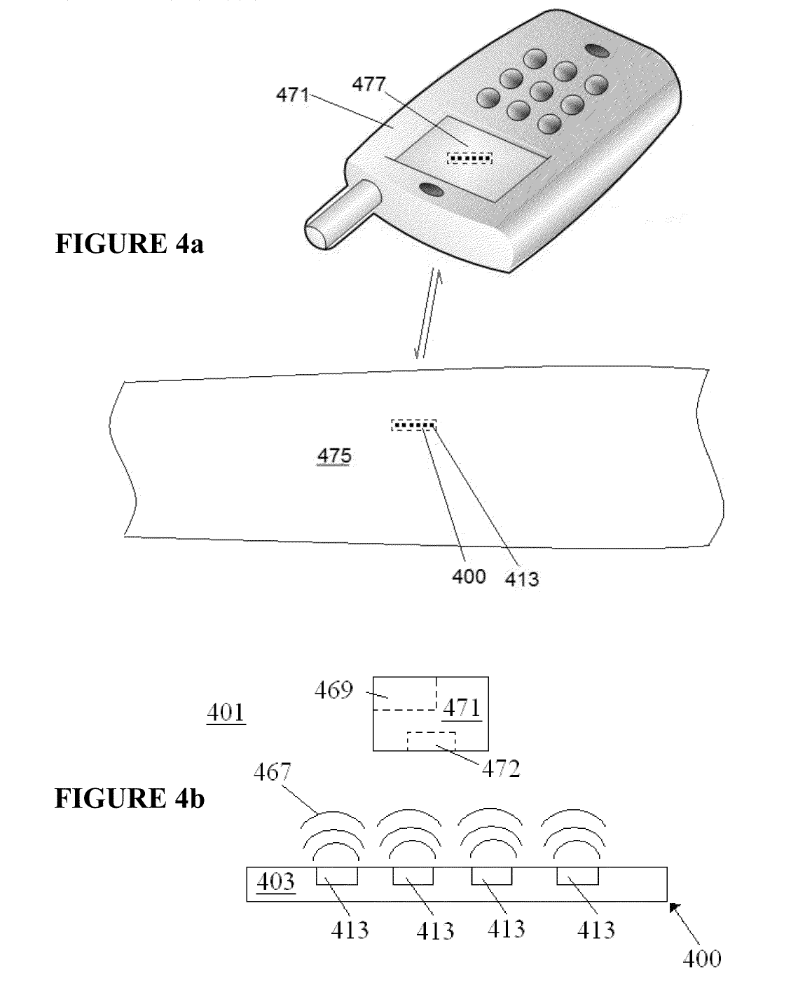

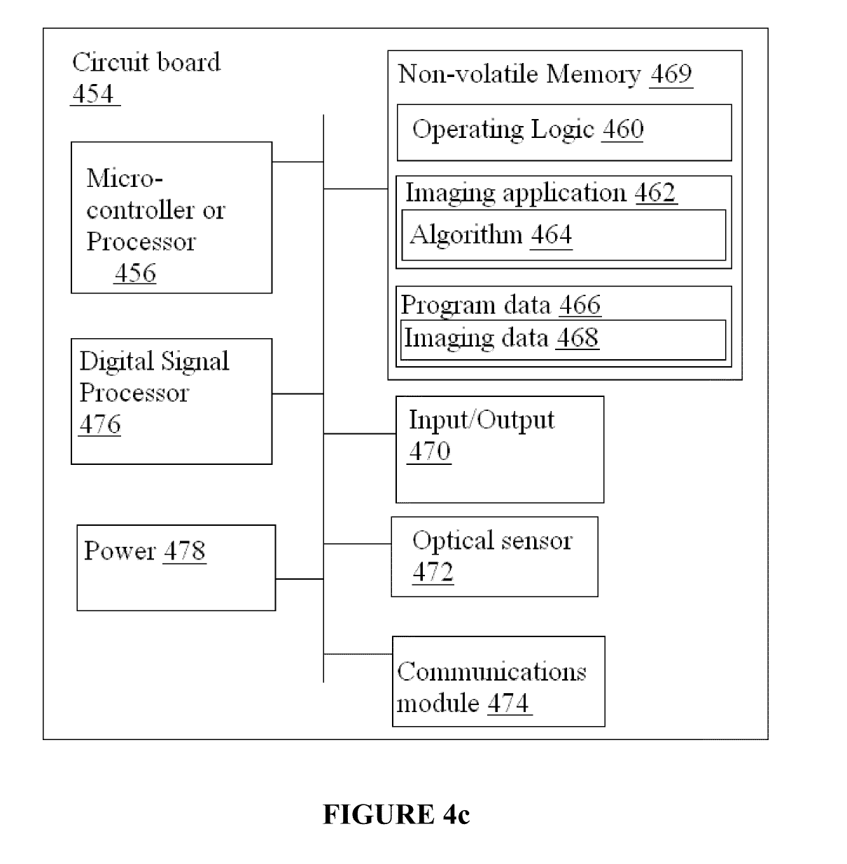

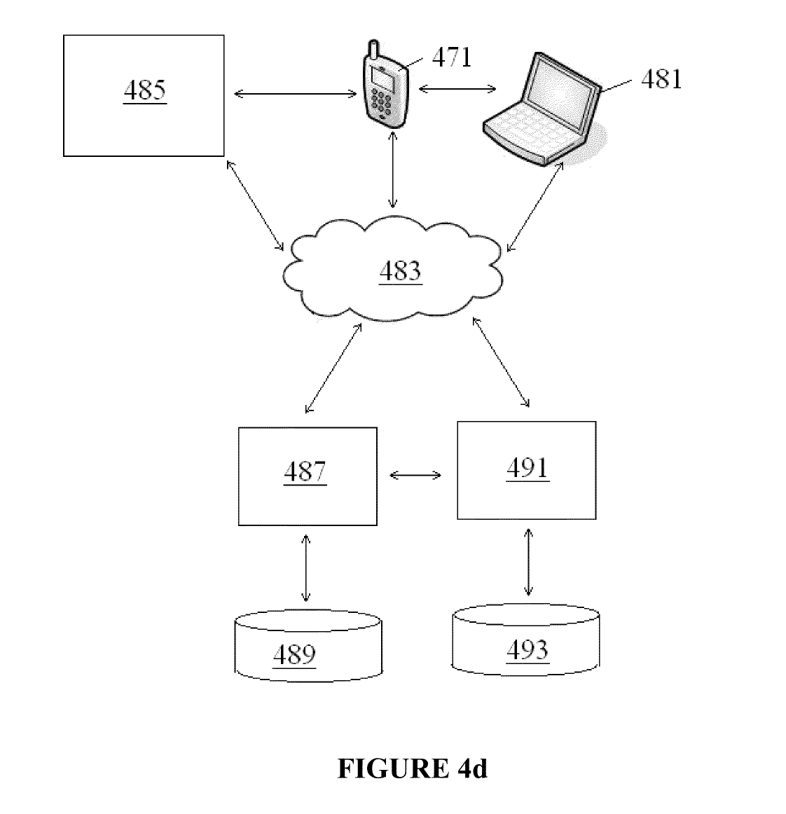

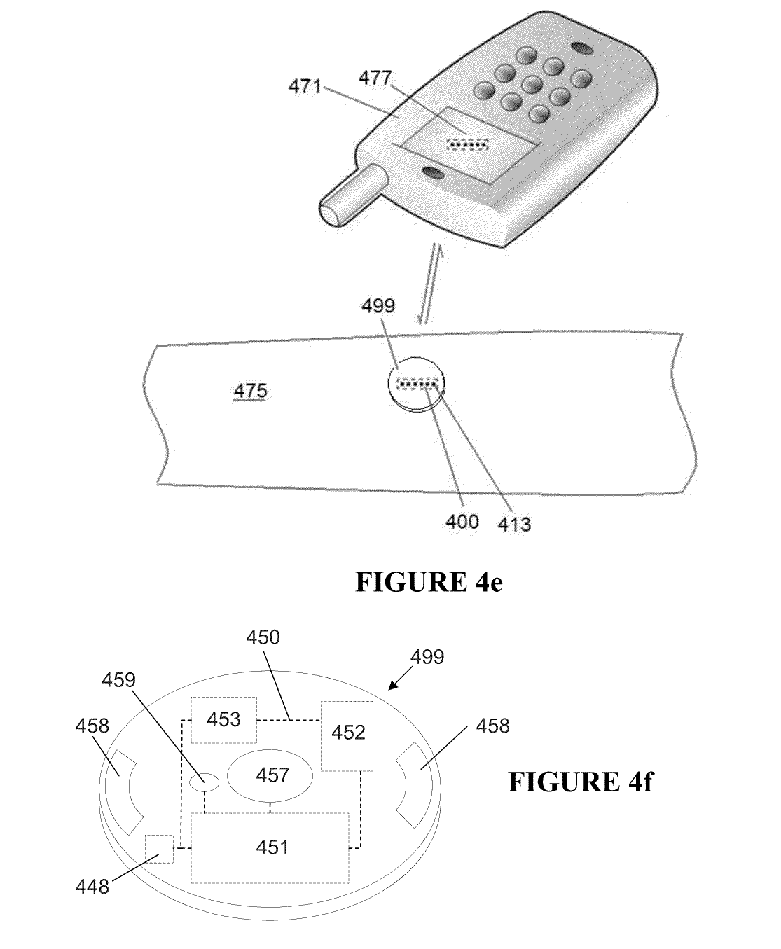

FIGS. 4a-f illustrate examples of mobile analyte monitoring systems and components thereof, in accordance with various embodiments. FIG. 4a illustrates the use of a reader device (e.g., electronic device 471) to capture an image of an implantable sensor 400 (shown implanted into a portion of the user's dermis 475). FIG. 4b shows a box diagram of a reader device and implantable sensor. FIG. 4c illustrates a circuit board of a cell phone configured for use as a reader device. FIG. 4d illustrates an example of a mobile analyte monitoring system that includes one or more additional computing devices, as discussed further below. FIG. 4e illustrates another embodiment of a mobile analyte monitoring system in which a reader device includes an electronic device 471 and a separate image capture device 499.

Examples of reader devices include, but are not limited to, personal electronic devices such as cell phones, smart phones, personal digital assistants (PDAs), tablet computers, laptop computers, media players, and other such devices. In particular embodiments, a reader device or a component thereof (e.g., image capture device 499) may be a mobile electronic device.

A reader device may be a single device, as described further below and illustrated by way of example in FIG. 4a. Alternatively, a reader device may include two or more devices communicatively coupled, as illustrated by way of example in FIGS. 4e and 4f. Therefore, in some embodiments, a “reader device” may include two or more electronic devices, and operations described and attributed herein to a reader device may be performed collectively by the two or more electronic devices.

In some embodiments, a reader device can include both a personal electronic device (shown in FIGS. 4a and 4e as 471) and an image capture device (shown in FIGS. 4e and 4f as 499) that is configured to be worn on, or otherwise attached to, a user's body (e.g., on an area of skin overlying an implantable sensor) during use. For example, image capture device 499 may be retained on the skin of the user over a sensor implantation site by an adhesive between the skin and the image capture device 499. Alternatively, image capture device 499 may be retained on the skin over a sensor implantation site by a belt, a band (e.g., worn in the manner of a wristwatch or armband), or an adhesive layer disposed over the image capture device 499 and portions of the surrounding skin. In one embodiment, image capture device 499 may have a clear or translucent portion (e.g., along the outer periphery) to allow light to pass through to the underlying analyte sensor. Alternatively, image capture device 499 may include a LED light or other light source that can be used to illuminate an underlying implanted analyte sensor. The LED light or other light source may be selectively illuminated at times that coincide with the capture of analyte sensor images by image capture device 499. In other embodiments, the LED/light source may remain continuously illuminated.

In various embodiments, the image capture device 499 may be configured to communicate data to a mobile electronic device such as a smartphone or a cell phone, or to another type of electronic device. The image capture device 499 and the personal electronic device 471 may each be configured to perform some of the reader device functions described throughout the present disclosure.

In a specific example, image capture device 499 may include one or more of a processor 451, an optical sensor 457, a memory 452, and a communications module 453 (e.g., a transmitter, transceiver, or other type of communications device) coupled by circuitry 450 (FIG. 4f). Optionally, image capture device 499 may include a power source 448 (e.g., a rechargeable battery or a replaceable battery). In some embodiments, image capture device 499 may be provided with an adhesive 458 for attaching the image capture device to the skin of the user. Optionally, image capture device 499 may include a light source 459, such as a LED light.

The electronic device 471 may be configured to receive images from the attached device and to perform some or all of the other processing and/or communications functions described herein for reader devices. The image capture device 499 may be configured to capture images of an implanted analyte sensor continuously, at predetermined intervals (e.g., every 10 seconds), and/or in response to a command from another device or system (e.g., electronic device 471, a manufacturer's computing system, a health management system, etc.). Image capture device may be operable to transfer captured images of the analyte sensor to electronic device 471 for analysis. Optionally, the image capture device 499 may be configured to perform a rudimentary image analysis to determine whether the captured image is satisfactory, and/or to transmit image data to the cell phone or other electronic device for analysis/further transmission. The reader device may thus collect, analyze, generate, and/or communicate analyte data or other health parameter data without requiring the intervention of the user to capture images of the implanted analyte sensor.

Image capture device 499 and electronic device 471 may be used in combination as a reader device under a variety of circumstances, such as for analyte monitoring while the user is asleep, for closely monitoring users who are brittle diabetics and/or have relatively large target analyte fluctuations requiring close monitoring, for greater convenience to the user (e.g., to get continuous results without requiring the user to manipulate the reader device in order to capture analyte sensor images), or to provide continuous data for controlling a medical device such an insulin or glucagon pump/delivery system. In a particular example, the image capture device 499 may be 0.5 inch in width/diameter, or 0.5-1.0 inch in width/diameter, and ≦0.25 inches thick.

Referring to FIG. 4c, a reader device may be a wireless mobile phone with one or more of the following features: circuit board 454, microcontroller or processor 456, digital signal processor 476, power module 478, non-volatile memory 469, input/output 470, optical sensor 472, and communications module 474. Communications module 474 can include a RF transmitter/receiver. Optionally, communications module 474 may also be configured to transmit and/or receive infrared (IR) or other signals.

Optical sensor 473 may be configured to detect electromagnetic radiation 467 that is reflected, deflected, and/or emitted from sensor 400. Optical sensor 473 may be any type of image capture device suitable for use in a personal electronic device. Optical sensor 473 can be, but is not limited to, a charge-coupled device (CCD) image sensor, a complementary metal-oxide-semiconductor (CMOS) image sensor, a CCD/CMOS hybrid sensor (e.g., sCMOS sensor), a Bayer sensor, a Foveon X3 sensor, a 3CCD sensor, and an active-pixel sensor (APS). Optionally, optical sensor 473 may be provided with one or more color filters, which may be removable/interchangeable.

Non-volatile memory 469 may include operating logic 460 (e.g., an operating system), imaging application 462, and program data 466. Imaging application 462 may include programming instructions providing logic to implement image analysis functionalities described herein. Program data 466 may include imaging data 468, as well as reference tables/values, reference images, previously determined representative values, and/or other data. Imaging data 468 may include previously captured images of implantable sensor 400 or corresponding image data.

Imaging application 462 can include one or more algorithms 464 for image analysis, calculation of representative values for analytes, tracking of representative values over time, analysis of a user's medication, and/or other functions. For example, imaging application 462 may include an algorithm 464 configured to analyze the effect of a user's medication based user inputs (e.g., times and dosages at which a medication was taken) and the determined concentrations of the medication or a related analyte at particular time points. Optionally, imaging application 462 may track the effect of the medication as a function of dosage and/or time, or suggest modifications in the dosage of the medication based on the analysis.

Microcontroller or processor 456 may be configured to operate optical sensor 472 and to execute operating logic 460, including imaging application 462. Operating logic 460 may include an operating system (OS). Input/output 470 may be configured to generate input signals in response to a user selection of a control, such as a keypad key or touchscreen/touchpad. Communications module 474 may be configured to transmit and receive communication signals for a call/audio message, an email message, and/or a text message. Communications module 474 may be a radio frequency transceiver, and may support one or more of any of the known signaling protocols, including but not limited to CDMA, TDMA, GSM, and so forth. Except for the manner in which any of the illustrated features, such as microcontroller or processor 456, are used in support of image capture/analysis functionalities as described herein, these features may otherwise perform their conventional functions as known in the art.

Referring now to FIGS. 4a and 4b, implantable sensor 400 may include one or more analysis regions 413 configured to detect a particular analyte within a given concentration range (i.e., the detection range), as discussed above. The responses of the analysis regions 413 may be detected through the overlying dermis of the user. A user may hold reader device 471 near the area of dermis 475 where implantable sensor 400 is located. The user may operate reader device 471 to capture an image of that area using optical sensor 473. Reader device 471 may execute imaging application 462 to determine the concentrations of target analytes in the interstitial fluid based at least on the captured image.

Optionally, the reader device may be configured to access a look-up table from program data 466 or a database (see e.g., FIG. 4d, databases 489/493) that stores one or more of a pre-determined pattern, reference image, and/or ranges for some or all of the pre-determined analysis regions. The reader device may then determine or calculate a representative value for an analyte based on the image data and corresponding detection ranges. In some examples, the reader device may select an analysis region that differs from a pre-determined pattern in size/area, contour, and/or location. The reader device may extrapolate a detection range for this analysis region based at least on the difference(s) between the selected and pre-determined pattern, and corresponding detection range(s) provided in the look-up table or database.

The concentrations/representative values, captured image, image data, and/or other relevant data (e.g. time, date, identity of analyte, etc.) may be stored in non-volatile memory 469 as program data or imaging data. Reader device 471 may track the concentrations/representative values over time, recording them in a table or other format that can be displayed or communicated to the user. Optionally, reader device 471 may display the captured image and/or determined representative value on a display 477, communicate the results to the user or to another device/system, and/or generate and communicate a message, notification, alert, instructions, or a representative value (e.g., a target analyte concentration, a temperature, a pressure, etc.) to a user of the reader device in a visual, audio, and/or tactile (e.g., vibratory) format. Optionally, the reader device may alert the user of a possible sensor malfunction, or that the sensor is approaching or has reached or exceeded the end of its recommended duration of use.

In some embodiments, the reader device may transmit a message, notification, alert, instructions, or a representative value (e.g., a target analyte concentration, a temperature, a pressure, etc.) to a medical service provider or caretaker. As shown in FIG. 4d, reader device 471 may be communicatively coupled to one or more computing devices or systems via a wireless connection or network. Reader device 471 may exchange data with one or more of a personal computer 481, a network 483, a medical device 485, a first computing system 487, a first database 489, a second computing system 491, and/or a second database 493. In some examples, first computing system/database 487/489 may be a medical provider or health monitoring computing system/database, and may be operated or accessible by a first medical provider, such as a primary care physician of the user. Second computing system/database 491/493 may be operated by a caretaker or a second medical provider such as a doctor's office, hospital, emergency medical service, or subscription-based service that notifies a medical provider of a potential emergency. Alternatively, second computing system/database 491/493 may be a computing system/database of a manufacturer of a sensor that can be read by reader device 471 (e.g., an implantable sensor). As described further below, the computing system of the manufacturer may analyze or track data received from the reader device to assess sensor performance. Medical device 485 may include an insulin pump that is worn by the user or implanted into the user's body. Alternatively, medical device 485 may include a dialysis machine and/or a glucagon delivery system.

In some embodiments, an analyte sensor may be read by a user without the use of a reader device. For example, the user may determine an approximate analyte concentration by viewing the analyte sensor without the aid of a reader device. Optionally, the user may be provided with a visual aid such as a chart, color key, or the like. The user may compare the response(s) of the analysis region(s) to the chart to determine an approximate analyte concentration. Alternatively, the user may interpret the response(s) of the analysis region(s) without the use of a visual aid. For example, after a period of time, the user may have sufficient experience with the use of the sensor to correlate the visible color change to an approximate analyte concentration. As another example, an analyte sensor may have multiple analysis regions that are configured to exhibit responses to the same analyte, but have different ranges of detection, such that at a given analyte concentration at least one of the analysis regions exhibits minimal or no color change. Based on which of the analysis regions displays a response, the user may determine that the analyte concentration is within a particular range.

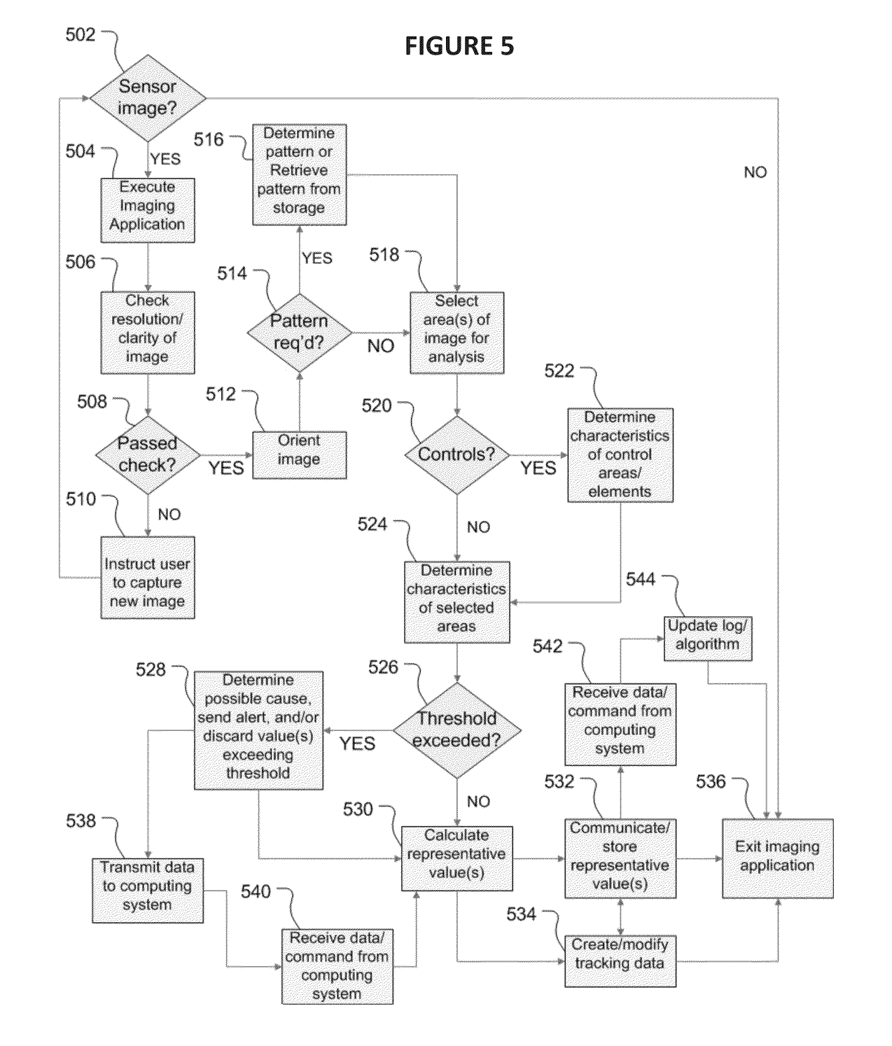

FIG. 5 illustrates a non-limiting example of a process for monitoring an analyte using a mobile analyte monitoring system, in accordance with various embodiments. In particular, such processes may be used with embodiments of a mobile analyte monitoring system described herein (e.g., system of FIGS. 4a-f). Image analysis processes, tasks/steps, sequential orders in which steps/tasks are performed, and distribution of analysis tasks among the reader device and other devices/computing systems may vary among embodiments. Many variations and modifications to the illustrated logic flow will be readily understood by persons with ordinary skill in the art in light of the present disclosure, which encompasses all such variations and modifications.

In addition, although the reader device is typically used to capture images of the sensor, one or more of the other functions described herein as being performed by the reader device may instead be performed by another device or system, such as a computer, database, medical device, etc., and vice versa. For example, the reader device may capture an image of the sensor and transmit the image data to a computing system for analysis. Alternatively, image analysis functions may be divided among the reader device and another device or computing system. For example, the reader device may be configured to determine a representative value for a target analyte and the computing system may be configured to track the representative values over time and/or to generate and send instructions to the reader device to adjust one or more operational parameters (e.g., to adjust a setting of the optical sensor, to capture another image, or to set a time at which another image should be captured).

Optionally, the illustrated logic may begin at block 502. At block 502, the reader device may determine whether a captured image is an image of an implantable sensor. In some examples, the determination may be based on an image recognition/matching process as is known in the art for machine recognition of human faces, structures, or items. In other examples, the determination may be based on input entered into the user device by a user, such as a user selection of a physical or virtual key, a button, an icon, or item on a displayed list. In still other examples, the reader device may determine that a captured image is an image of an implantable sensor in response to determining that a filter has been coupled to or engaged within the optical sensor of the reader device. In some embodiments the reader device may determine that a filter or lens has been attached to the optical sensor/reader device, or that a filter or lens is needed to improve or ensure image quality. Optionally, the reader device may instruct the user to attach a filter/lens, and/or confirm that the filter/lens has been attached properly such that the user can proceed to capture the image of the analyte sensor. Alternatively, some sensors may include a bar code or other identifier that can be used by the reader device to determine that the sensor was calibrated by the manufacturer (e.g., during or after production of the sensor). The reader device may determine that a captured image is an image of an implantable sensor based at least in part on the bar code or other identifier.

Alternatively, in some embodiments, the reader device may determine that a captured image is an image of an implantable sensor based on the configuration of the analyte sensor. For example, in some embodiments, the reader device may identify one or more features of an analyte sensor (e.g., analysis regions, control regions, chambers, control elements, body, and/or base) and identify the analyte sensor as a particular type/model based on the position, size, spacing, and/or general configuration of the identified feature(s). The reader device may then use/select an image analysis algorithm (e.g., for calibrating and/or reading the analyte sensor) that corresponds to the identified type/model. Thus, in some embodiments, the configuration of the analyte sensor may be “read” and used by the reader device in the same or similar manner as a bar code.

Some embodiments may lack a block 502, and may execute the imaging application either automatically (e.g., in response to activation/use of the optical sensor) or upon receiving an input by the user to display/execute the imaging application or some portion thereof.

At block 504, the reader device may execute the imaging application (e.g., imaging application 462) in response to user input, a determination that a captured image is an image of an implantable sensor, or other factor (e.g., a pre-determined time for capturing an image of the implantable sensor, a received command from a remote device/computing system of a caretaker or medical care provider, etc.).

Optionally, at block 506, the reader device may check the captured image to determine whether the quality of the image is sufficient for analysis. This may be based on one or more pre-determined thresholds or limits for resolution, brightness/contrast, exposure/dynamic range, one or more settings or operation parameters of the optical sensor (e.g., shutter speed, exposure setting, Exposure Value Compensation setting), or other factors. If the reader device determines at block 508 that the quality of the image is insufficient for analysis, the reader device may communicate an instruction to the user to capture another image of the implantable sensor (block 510). The instruction may include a recommendation for the image capture, such as a recommended adjustment or setting for the optical sensor or a distance at which the optical sensor should be held from the implantable sensor for image capture. Optionally, the reader device may be configured to indicate whether one or more conditions (e.g., a current position or orientation of the reader device, lighting conditions, or a distance of the reader device from the sensor), is acceptable for capturing an image of the sensor, based at least on a bar code or other identifiers provided on the sensor. If the reader device determines that the quality of the image is sufficient for analysis, the logic may proceed to block 512.

At block 512, the reader device may orient the image. In some examples, this may involve rotating, resizing, and/or centering the image. In some embodiments, the reader device may orient the image according to one or more control elements that are located on or within the sensor (see e.g., FIG. 1d, control elements 299). For example, the reader device may orient an image of the sensor of FIG. 1d by aligning the four illustrated control elements 299 against a pre-determined pattern, a previous image, or pre-determined shape (e.g., four opposite corners of a square, or the top, bottom, left, and right sides of a square). Optionally, the reader device may use a bar code or other identifier of the sensor to retrieve calibration data from a computing system, network, or cloud. The reader device may thus orient the image based on the bar code or other identifiers.

Optionally, the reader device may determine that a pattern is required to orient or analyze the captured image (block 514), and may retrieve the pattern from local storage (e.g., program data 466 or imaging data 468) or from a remote database (block 516). In some examples, block 514 may be absent, and the reader device may automatically retrieve a pattern in block 512 for orientation of the image, orient the image without a pattern (e.g., by way of reference to control elements, other features of the sensor, or implantation site), or proceed without orienting the image.

At block 518, the reader device may select one or more areas of the image for analysis. The reader device may select portions of the captured image that correspond to analysis regions (e.g., analysis regions 413) based on color, intensity of emission, distance from the center/edge of the base/body or other feature, location on the sensor, prior readings, programmed instructions, and/or a pre-determined pattern or reference image. For example, the reader device may select areas of a captured or real-time image based at least on a pre-determined pattern (e.g., a layout of the analysis regions/chambers of the implantable sensor). In other examples, the reader device may select areas based on position/distance relative to one or more control elements. Optionally, the reader device may increase or decrease the size of a selected area based on image resolution (e.g., select a larger area where image resolution falls below a minimum threshold).

At block 520, the reader device may select one or more areas of the image for use as controls. Optionally, the reader device may determine whether or not control areas should be selected for analysis, based on factors such as image quality and pre-stored information about the implantable sensor. In other embodiments, the reader device may select control areas in block 518 before, during, or after the selection of areas for analysis (e.g., areas corresponding to analysis regions). In any case, selected control areas may correspond to control regions (e.g., duplicate analysis regions configured to detect the same analyte within the same detection range, or analysis regions configured to detect non-target analytes or other parameters), control elements, other features of the sensor, and/or features of the overlying or proximal dermis or implantation site. Again, the selection of control areas may be based on a pre-determined pattern or any of the other factors described above with regard to selection of analysis areas.

Optionally, at block 522, the reader device may determine one or more characteristics of the selected control areas of the captured image before proceeding to block 524, in which other selected areas are analyzed (e.g., areas corresponding to analysis regions configured to detect the target analyte(s)). In other embodiments, blocks 522 and 524 may be reversed in order. In still other embodiments, blocks 522 and 524 may be combined into a single block. In any case, the determined characteristics of the selected control areas may include, but are not limited to, a color value, an intensity/brightness value, a size/dimension or position/orientation value, and/or a value representing a difference between any of the above values and a threshold or reference value.

At block 524, the reader device may determine one or more characteristics of one or more remaining selected areas of the captured image. Again, the determined characteristics may include, but are not limited to, a color value, an intensity/brightness value, a size/dimension or position/orientation value, and/or a value representing a difference between any of the above values and a threshold or reference value.

In some embodiments, these values may be calculated based at least in part on the values determined in block 522. For example, the reader device may determine at block 522 a value that represents a difference between the color of a control element (e.g., a colored spot) prior to implantation of the sensor and the color of the control element in the image of the implanted sensor (i.e., a “post-implantation color change” value). This value may be used in the determination of a color value of an analysis region to correct for, or minimize the effect of, factors such as implantation depth, skin tone, dermal translucence, lighting conditions, and/or others.

In some embodiments, one or more of the values determined for a control area may be used in the determination or correction of a value for another control area. Continuing the above example, a value that represents a difference between the color of the control element before and after implantation may be used to determine or correct a color value for a selected control area that corresponds to a control region configured to detect a non-target analyte. The non-target analyte may be one that is typically present at relatively constant levels within the dermis (e.g., sodium, pH, uric acid, chloride, potassium, or cholesterol). The reader device may determine a color value for the corresponding area of the image and adjust the color value based on the post-implantation color change value to correct for, or minimize the effect of, skin tone and other factors as discussed above.

Optionally, at block 526 the reader device may compare one or more of the determined values to one or more threshold values and determine whether any of the determined values exceed the threshold value(s). A threshold value may be, for example, an upper or lower limit of a pre-determined range of values, a determined value of a control region or analysis region, or an upper or lower limit of a range of values determined by the reader device based at least in part on one of the other determined values (e.g., a range of values representing the determined value of a control area and higher/lower values within a pre-determined margin of error). In some examples, the reader device may retrieve a standard or customized set of threshold values from a local or remote storage. In other examples, the reader device may determine the threshold values based on one or more previous readings. The reader device may apply different threshold values to at least some of the determined values. For example, the reader device apply a first set of threshold values to a value determined for an area corresponding to a potassium-detecting analysis region, and apply a second set of threshold values to a value determined for an area corresponding to a control element such as a colored spot. The reader device may also apply a set of control values to more than one determined threshold value (e.g., a maximum intensity threshold, above which results may be unreliable).

If the reader device determines that one or more of the determined values exceeds an applied threshold value, the reader device may generate a response at block 528. Examples of responses include determining a possible cause for the difference, sending an alert (e.g., to a user, a caretaker, a medical provider, etc.), transmitting data to a network, system, or remote computer/database, and/or discarding the value(s) that exceeded the applicable threshold. Determining a possible cause may include actions such as accessing prior readings from a memory/storage, checking one or more settings of the optical sensor, assessing the number or percentage of determined values that exceed the corresponding thresholds, or checking a reader device log for system errors. Sending an alert may include actions such as communicating an auditory (e.g., ring tone or alarm tone), vibratory, and/or visible message (e.g., text, email, display, or light activation) to the user, a caretaker, or a medical facility. Optionally, an alert may be sent to a medical device, such as an insulin pump, in the form of an instruction or command to adjust the operation of the medical device.

In some embodiments, the reader device may transmit image data, determined characteristics/values, or other relevant data to a network, system, or remote computer/database (block 538). For example, the reader device may transmit such data to a computing system of the sensor manufacturer. The computing system of the manufacturer may remotely monitor sensor performance in this manner, using data received from the reader device to analyze and track sensor performance characteristics for quality control purposes.

Optionally, at block 540, the reader device may receive data and/or a command from the computing system of the manufacturer. The data/command may be an update to the reader device (e.g., to an algorithm), or an alert for the user with regard to the functionality of the device. For example, the received data/command may be a message instructing the user to recalibrate the sensor (e.g., with another testing method, such as conventional glucose strips to check glucose levels) or advising the user that the sensor should be replaced within a particular timeframe. As another example, the data may include a revised or updated algorithm configured to offset the effects of sensor deterioration/wear/aging (i.e., to offset a reduction in the magnitude or degree of the sensor's exhibited response to an analyte concentration as the sensor ages). In some examples, the computing system of the manufacturer may use data from one or more sensors to determine a projected useful life of a sensor, such as 30, 60, 90, 180, or more than 180 days.

At block 530, the reader device may determine a representative value for an area of the image corresponding to a portion of an analysis/control region. The determination may be based at least in part on the value determined for that area in block 522/524. In some embodiments, the value determined may be averaged with one or more other values (e.g., averaging determined values for duplicate or triplicate analysis regions, or averaging multiple determined values for different areas of the same analysis region).

To determine the representative value, the reader device may first determine the identity of the target analyte and the detection range of the corresponding portion of the analysis/control region. These values may be stored locally or remotely in the form of a look-up table or other record. In some examples, the reader device may refer to a pre-programmed sensor layout in order to determine a detection range for an area based on its position relative to one or more of control elements, the center or edge of the sensor, and/or another feature of the sensor.

In some examples, a look-up table/record or portion thereof may include representative values for each analysis area and/or portion of the implantable sensor. The data may be organized in various ways, such as in a single table for the entire sensor or in separate tables for each analyte/detection range. In any case, the look-up table/record(s) may have a list of possible color values, intensity values, or sub-ranges of such values, each associated with a corresponding representative value. Thus, the reader device may determine the representative value by accessing the record/table (or portion thereof) for the relevant portion of the implantable sensor, locating the color value/intensity/sub-range that matches or most nearly matches the determined value, and retrieving the representative value associated with that color value/intensity/sub-range.

Alternatively, the reader device may be provided with a formula for calculating a representative value for a given area of the image. This may be done, for example, to adjust the representative value based on one or more of the control area/element values. A different formula may be provided for each analyte/detection range or combinations thereof, or for each analysis region of the sensor. The relevant formulas may be stored locally or remotely, and accessed by the reader device as needed.

Calculating a representative value may include comparing the representative value to one or more reference values. Some reference values may be pre-determined, such as a glucose range. Other reference values may be representative values of a control area. For example, a selected area may correspond to a control region configured to detect a non-target analyte typically present at relatively constant levels within the dermis (e.g., sodium, pH, uric acid, chloride, potassium, or cholesterol). The reader device may determine a representative value for the non-target analyte and compare the representative value to an expected range of values. If a difference between the reading and the reference values is determined to exceed a margin of error, the reader device may respond by adjusting one or more representative values (e.g., glucose values) as a function of the difference. Alternatively, the reader device may determine that the reading is inaccurate and disregard it, determine that the sensor is malfunctioning, and/or send an alert as discussed with regard to block 528.

Optionally, the reader device may compare determined values and/or representative values for two, three, or more than three selected areas of the image to determine whether a portion of the sensor is exhibiting a response that is inconsistent with the response of another portion of the sensor. The inconsistency may be, for example, a difference in response time, a difference in color, or a difference in intensity. The reader device may use the comparison to determine that the sensor is leaking or otherwise malfunctioning, determine a time frame for replacement of the sensor, or engage in error correction or data smoothing to determine a representative value. Optionally, the reader device may determine that a response or value from a region exceeds a predetermined threshold/value, differs from an average or other selected value by more than a predetermined limit, or is outside a particular range, such as an expected range. In response, the reader device may disregard that response or value when determining a representative value for the target analyte (or non-target control analyte).

Some control regions may be duplicates of analysis regions, configured to detect the same target analyte within the same range of detection and response. The reader device may compare the responses of the two regions and determine whether the responses are the same within a margin of error. If a difference between the responses is determined to exceed the margin of error, the reader device may determine that the sensor is malfunctioning, alert the sensor user, and/or disregard one or both responses. Alternatively, the reader device may average the responses from the two regions and determine a representative value for the target analyte (or non-target control analyte) based on the determined average.

Some analysis/control regions may be used by the reader device to correct or determine representative values for a target analyte based on a local condition such as local blood/fluid flow, or changes/differences in analyte diffusion rates. For example, a control region may be configured to detect an analyte that is administered to an animal. Optionally, the analyte may be administered to the animal simultaneously or contemporaneously with a dose of a drug, a treatment, or a target analyte. The reader device may determine the time at which the analyte is administered. The reader device may read the response(s) of the control region at pre-determined times, at timed intervals, or continuously. The reader device may then correct or determine a representative value for a non-target analyte as a function of the length of time between the administration of the drug/treatment and the detection of the analyte by the control region. Optionally, the reader device may determine that the length of time exceeds a predetermined limit and alert the sensor user or reader device user of a condition such as poor circulation or possible sensor malfunction.

The response time may be used to calibrate the reader device or adjust one or more representative values. For example, the reader device or a computing system may determine a sensor lag time based on the response time. The sensor lag time can be a difference between the length of time required for the sensor to detect an analyte (e.g., a drug, treatment, or other analyte) or analyte concentration change in the dermis and the length of time required for the analyte to be detected in an analysis of whole blood, plasma, or other fluid(s). The reader device may then adjust one or more of the representative values to correct for the lag time. In some examples, the reader device may be programmed to remind a user to capture an image of the sensor at particular times or intervals. The sensor lag time may be used by the reader device to adjust those times or intervals.

At blocks 532 and 534, the reader device may optionally communicate/store the representative values and/or create/modify tracking data, respectively, as discussed above. In one embodiment, a reader device may be configured to respond to a determination of a representative value as being above or below a predetermined threshold by transmitting the representative value to a computing system or device of a healthcare professional. The computing system or device may be programmed to respond by generating and sending a communication to the user (e.g., a phone call, text message, email message, etc.) to check the result using another analyte sensing device or system, such as a blood glucose meter.

Optionally, at block 532 the reader device may communicate calculated values and/or other data to another computing system, such as a computing system of a sensor/device manufacturer as described above with regard to block 538. At block 542, the reader device may receive data from the computing system, and may update a log and/or algorithm at block 544 as described with regard to block 540.

In another embodiment, at block 532 the reader device may communicate calculated values and/or instructions to a medical device. For example, the medical device may be an insulin pump, and the reader device may transmit a representative value of the user's glucose level to the insulin pump. Optionally, the reader device may generate and transmit one or more instructions to the insulin pump to increase or decrease the amount of insulin delivered to the user. Alternatively, the insulin pump may be programmed to adjust one or more operating parameters based on a representative value or other data received from the reader device. In some embodiments, the medical device may include a glucagon delivery system. The medical device may ping the reader device for data at timed intervals or in response to an event (e.g., input from a user or medical care provider system, or based on an algorithm of the medical device). In some examples, the medical device or a computing system of a medical care provider or sensor manufacturer may prompt the reader device to take a new reading of the sensor, or alert the user to take a new reading of the sensor. Alternatively, the computing system of a medical care provider or sensor manufacturer may send data to the medical device indicating a need for a particular number of readings at particular intervals/times, and the medical device may request the readings/data from the reader device or send a message to the reader device reminding the user to capture images of the sensor at those intervals/times.

At block 536, the reader device may exit the imaging application.

Imaging application 462 is one example of an application suitable for use with a mobile analyte monitoring system. As used herein, the term “imaging application” refers to a program that directs a processor to perform various tasks related to analyte monitoring (e.g., image analysis, calibration, tracking of data, etc.). Imaging applications and operations thereof may vary among embodiments. Optionally, an imaging application may include, or may be provided with, reference data such as reference tables/values, reference images, and/or other relevant data (e.g., program data 466). Some imaging applications may be developed or configured for use with a particular type of reader device (e.g., a smartphone or tablet computer) and/or operating system (e.g., Google Android, Apple iOS, Nokia Symbian, RIM BlackBerry OS, Samsung Bada, Microsoft Windows Phone, Hewlett-Packard webOS, Linux operating system). Again, these examples are provided merely by way of illustration, and imaging applications may be configured/adapted/developed for use with many other types of reader devices (e.g., tablet computer, personal digital assistant, camera) and/or operating systems. Some imaging applications may be “cross-platform” applications developed or configured for use with multiple types of reader devices/operating systems. In some embodiments, a reader device may an iPhone or an iPad.

In some embodiments, an imaging application may be pre-installed on the reader device (e.g., by the reader device manufacturer). In other embodiments, the application may be provided in a physical medium, such as an optical disc (e.g., a CD, a DVD), a data storage disk (e.g., a ZIP disk), a flash memory device (e.g., a USB flash drive, a memory card), and the like. Alternatively, the application may be downloaded/electronically transmitted to the reader device or associated computer system (e.g., the user's personal computer) over a network (e.g., the Internet). The application may be made available for download from a computer system or database of a third party (e.g., a manufacturer of the sensor, a manufacturer of the reader device, a medical service provider, a software developer, a software distributor, or a web-based application store, such as the Apple App Store). In some embodiments, the imaging application may be a web-based application that resides on a server of the third party and is accessible by the reader device via the Internet (e.g., as a web application). In one example, a portion of the web-based imaging application may be downloaded to the reader device and may reside on the reader device thereafter. Alternatively, a portion of the imaging application may be downloaded to the reader device each time the reader device accesses/uses the imaging application.

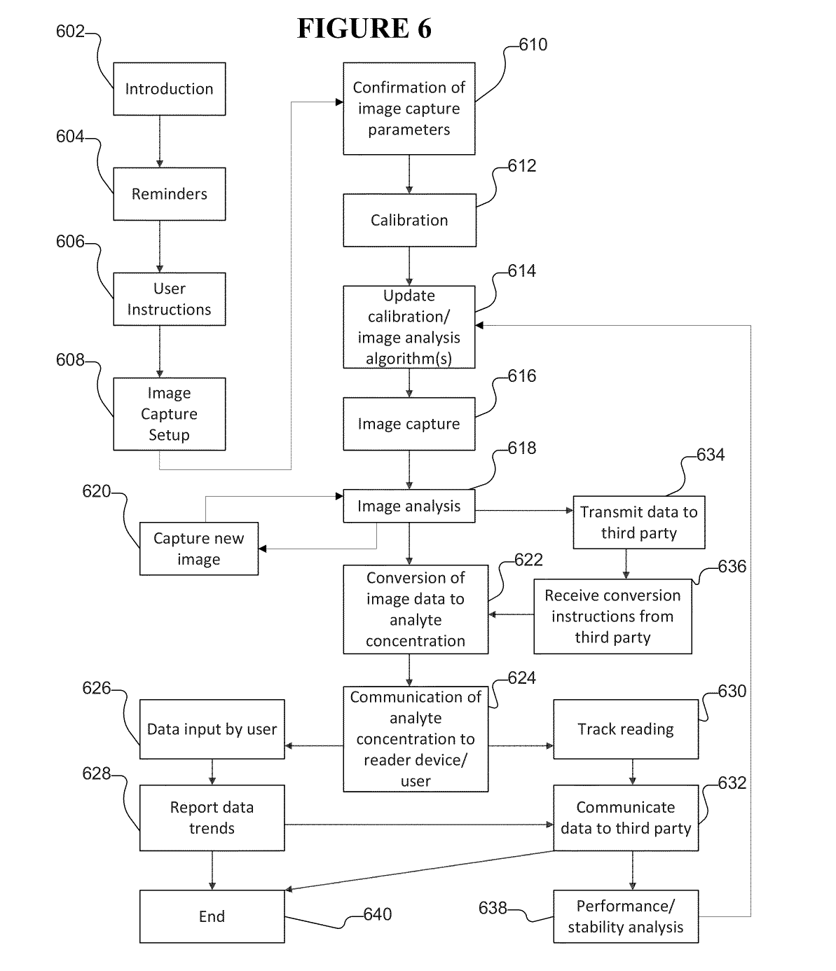

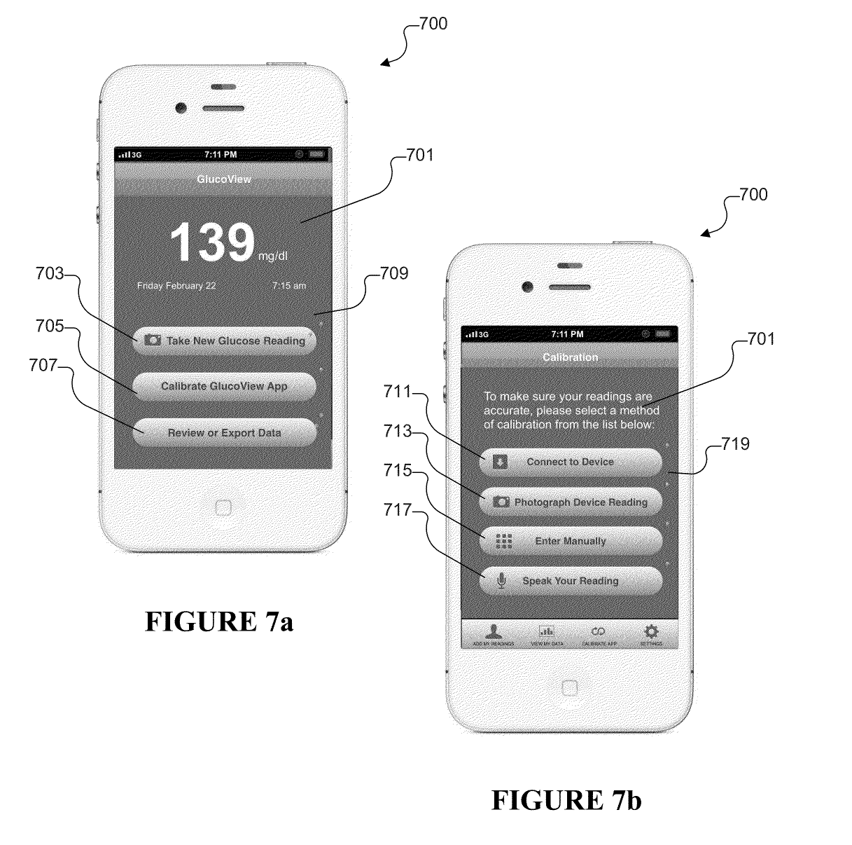

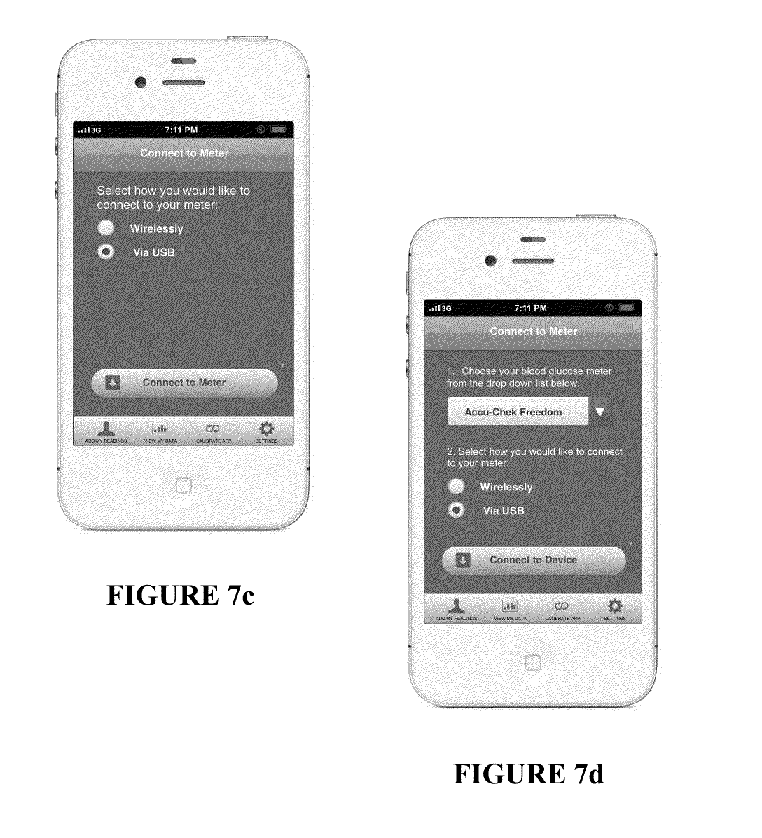

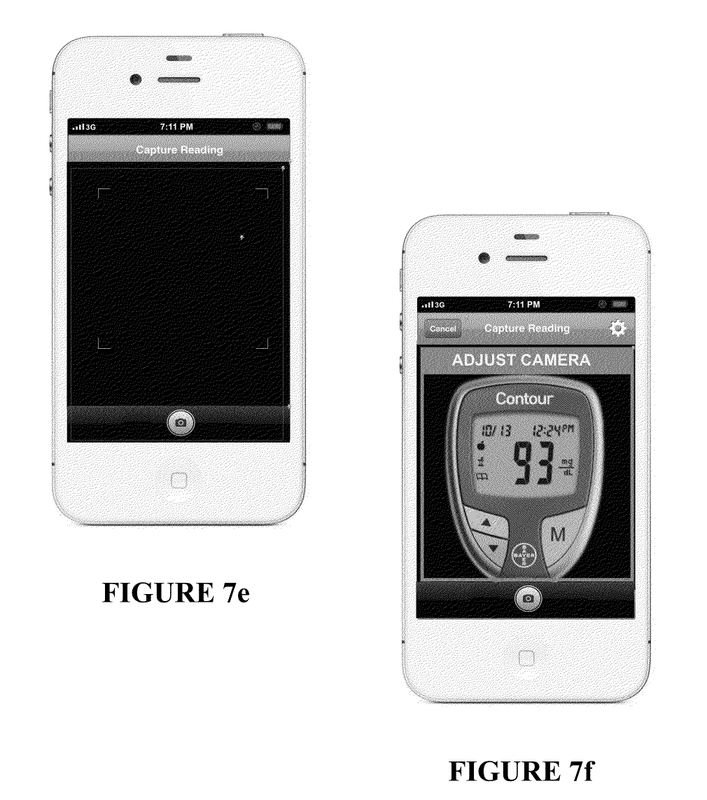

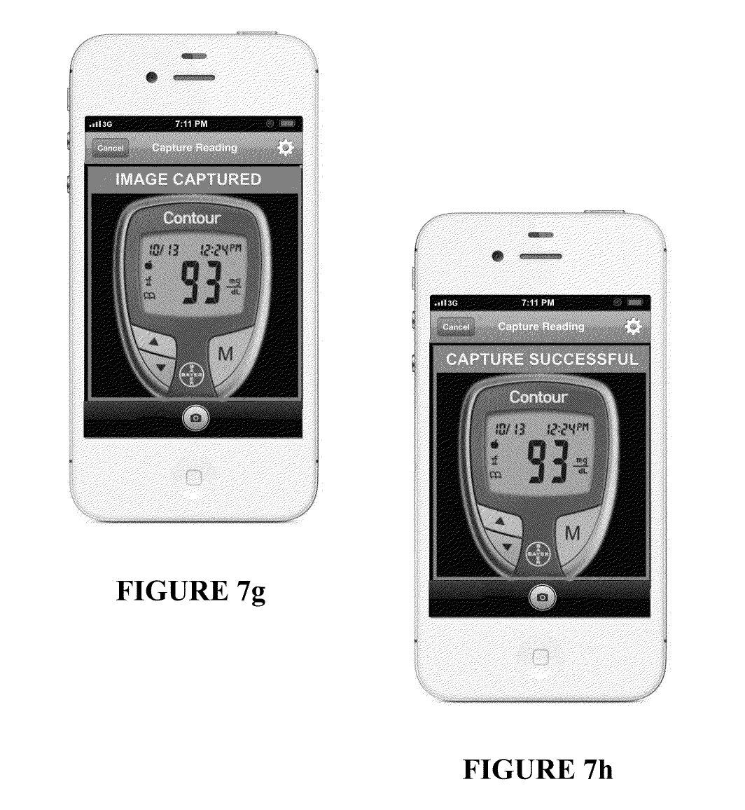

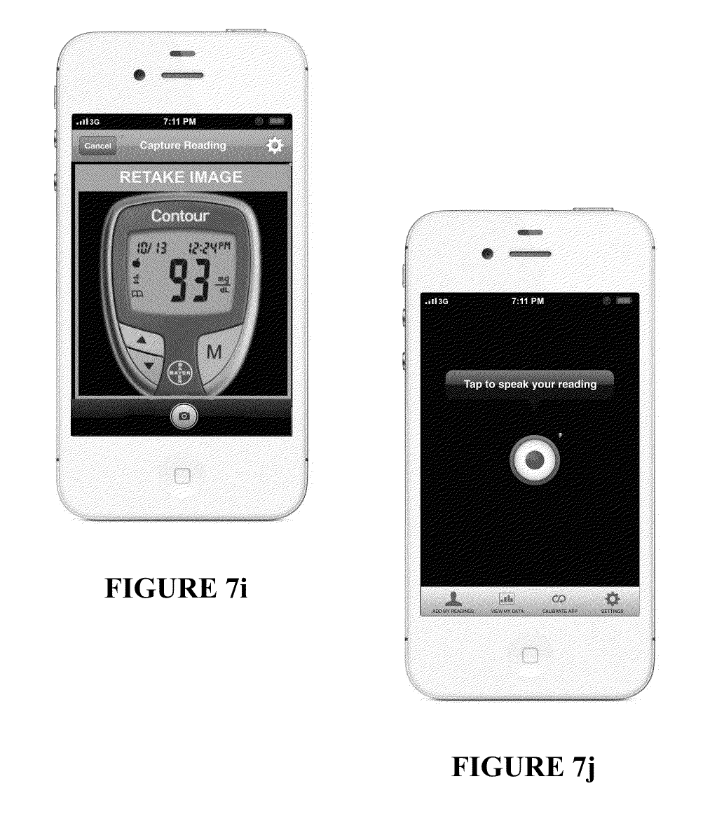

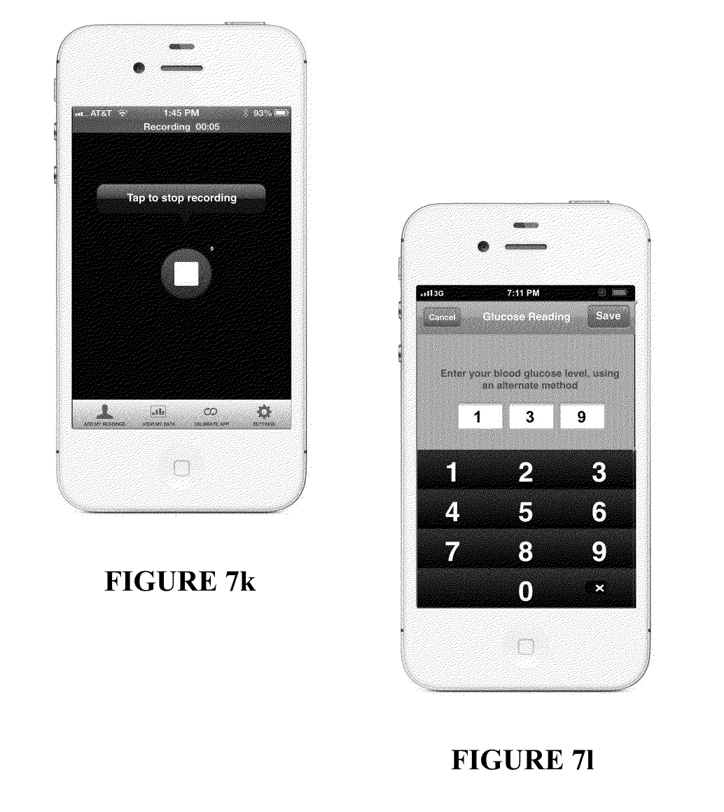

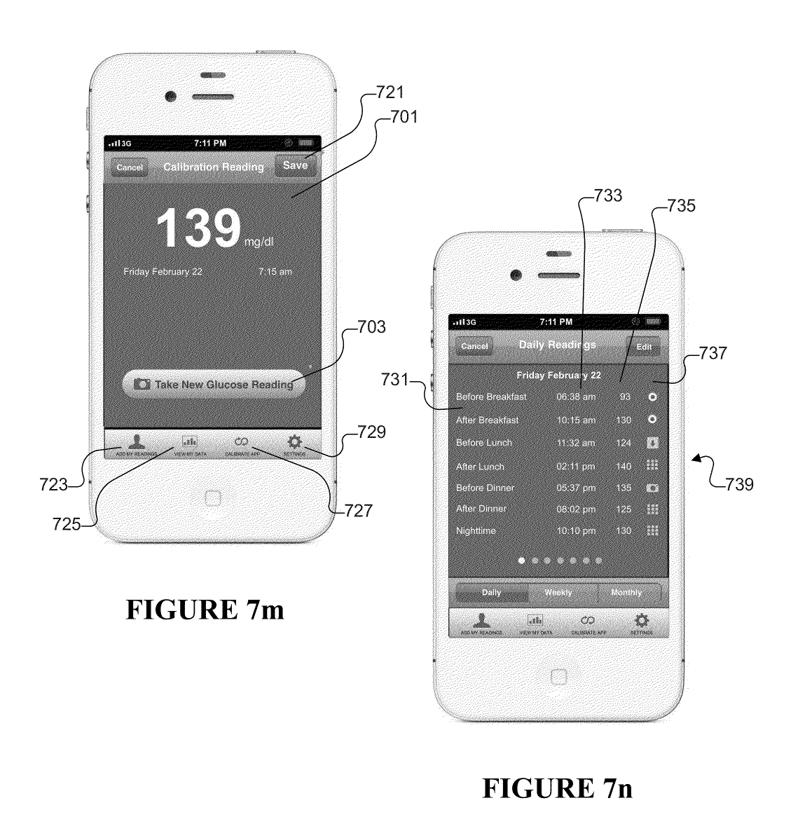

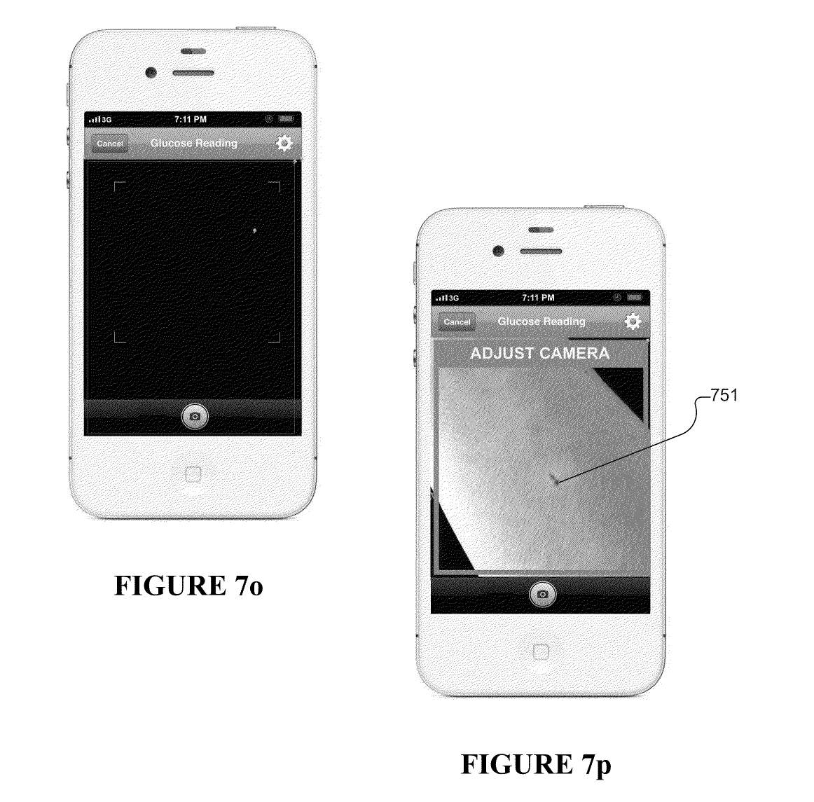

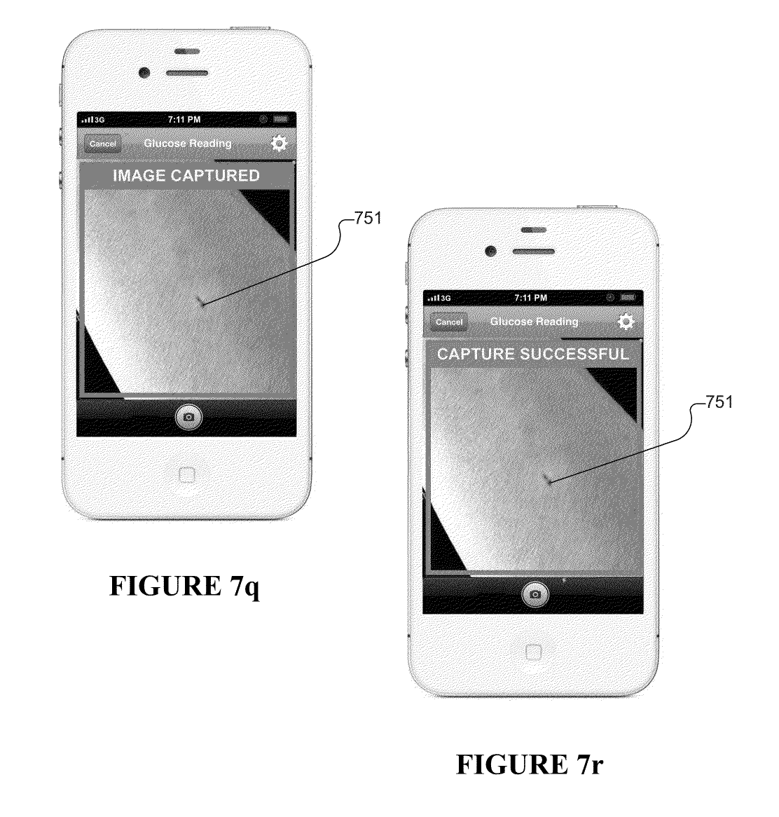

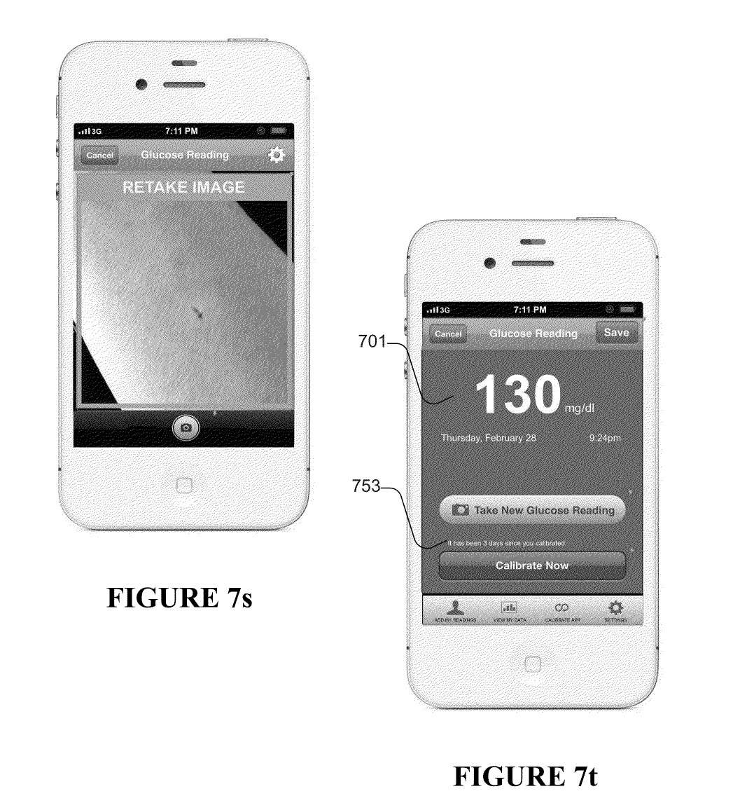

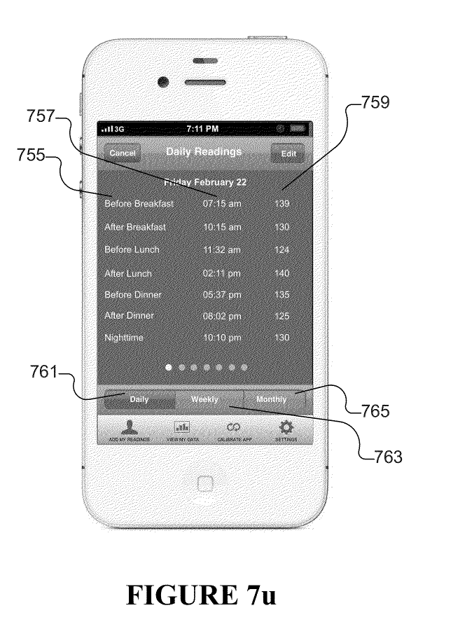

FIG. 6 illustrates a non-limiting example of a process for monitoring an analyte, in accordance with various embodiments. FIGS. 7a-7u illustrate examples of user interface displays corresponding to some operations of FIG. 6. As described further below, the process illustrated in FIG. 6 may include one or more of the operations illustrated in FIG. 5. Again, such processes may be used with embodiments of a mobile analyte monitoring system described herein (e.g., system of FIGS. 4a-f). In some embodiments, the illustrated processes may be, or may include, an algorithm of an imaging application.

Various operations, sequential orders in which operations are performed, and the distribution of operations among the reader device and other devices/computing systems may vary among embodiments. For example, in some embodiments, one or more of the operations may be performed locally by the reader device and others may be performed remotely by one or more third party computer systems. For the purposes of the discussion below, a third party computer system can be a computer system, website, database, server (e.g., a network server, a cloud server), or other digital distribution platform of a third party such as the analyte sensor manufacturer, a medical services provider, and/or an imaging application developer. Again, many variations and modifications to the illustrated processes and user interface displays will be readily understood by persons with ordinary skill in the art in light of the present disclosure, which encompasses all such variations and modifications.

Referring first to FIG. 6, at block 602, an overview of the imaging application may be provided to the user. In some embodiments, block 602 may be performed the first time that the imaging application is accessed by the user and/or loaded onto the reader device. The overview may include general instructions to the user for accessing or using the imaging application. Such instructions may be provided in visual and/or auditory form. For example, the instructions may be provided as a series of simulated user interface displays associated with sensor insertion, image capture, data entry, and/or calibration of the reader device. Optionally, block 602 may include a registration process or interface that allows the user to create a user profile and/or register the device with a third party server/website.

At block 604, reminders may be set for prompting the user to perform one or more tasks, such as calibrating or recalibrating the reader device, taking a sensor reading (e.g., capturing an image of the sensor), and/or entering user data (e.g., meal data, medication data, etc.). In some embodiments, reminders may be set by the sensor manufacturer. In other embodiments, reminders may be set by the user. In still other embodiments, reminders may be set by the user and suggested alterations to the reminders may be provided by a third party computer system based on data received from the reader device. For example, the third party computer system may determine based on one or more factors (e.g., user analyte concentration data, analyte concentration data from other users of analyte sensors from the same lot or batch, data from other users with similar medical parameters, clinical data, etc.) that the reminders should be more frequent, less frequent, or for different times/dates than those set by the user.

At block 606, the reader device may provide instructions (e.g., as a visual display and/or as auditory output) to the user for performing one or more actions such as capturing images, replacing the sensor, reader device calibration/recalibration, and/or blood glucose level confirmation. In some embodiments, the instructions may be provided at block 606 during the user's initial use of the imaging application. In one embodiment, at block 606 the reader device may provide instructions for inserting the analyte sensor (e.g., analyte sensor 100) into the user's dermis. For example, the instructions may include instructions for using an analyte sensor insertion device to insert the analyte sensor into the dermis of the user at a desired depth and orientation.

At block 608, one or more image capture parameters may be selected or determined. Image capture parameters may include, but are not limited to, a recommended reader device position for image capture (e.g., a distance at which the reader device should be positioned from the implanted sensor, an angle at which the reader device should be held), lighting conditions (e.g., minimum light levels, use of flash), image resolution, autofocus, and/or other imaging parameters. In various embodiments, the image capture parameters may be set based at least in part on variables such as the capabilities of the reader device (e.g., type of optical sensor, focal length, maximum image resolution, add-on filter/lens, etc.). In some embodiments, the reader device or a third party computer system may select some or all of the image capture parameters based on data such as reader device configuration and/or optical sensor type. In other embodiments, the reader device or third party computer system may select or adjust one or more of the image capture parameters based on one or more captured images. For example, a reader device with a light source may operate the light source to adjust lighting conditions based on a recently captured image.

At block 610, the image capture parameters may be confirmed. In some embodiments, the reader device and/or a third party computer system may confirm one or more image capture parameters by prompting the user to capture an image of an implanted analyte sensor, an intended sensor insertion site, or other target to confirm that exposure, distance, and alignment are correct. In some embodiments, the image capture parameters may be confirmed by the reader device. In other embodiments, the image capture parameters may be confirmed by the user or by a third party computing system. Optionally, at block 610 the reader device and/or third party computing device may analyze a captured image of the implanted sensor to determine whether the analyte sensor has been implanted correctly into the dermis (e.g., to the desired depth, at an intended or suitable insertion site).

In some embodiments, some or all of the selection/determination of image capture parameters (block 608) and/or confirmation of image capture parameters (block 610) may be performed automatically by the reader device, a third party computer system, or some combination thereof. For example, the reader device may automatically capture and analyze an image, adjust one or more of the image capture parameters based on the image, capture and analyze another image, re-adjust image capture parameter(s) based on that image, and repeat the capture-analysis-adjustment process as needed to optimize the image capture parameters. As another example, reader device may capture a series of images as a video (e.g., upon initiation by the user, or automatically). While the video is captured, the reader device may automatically adjust one or more of the image capture parameters (e.g., adjust operations of the optical sensor/camera). The reader device may be provided with one or more algorithms (e.g., a calibration algorithm, an image processing algorithm, or an image capture algorithm for determining capture parameters) and/or software configured to provide this capability. Therefore, in some embodiments block 608/610 may be performed by the reader device with minimal user interaction (e.g., the user may holding the reader device and/or initiate image capture).

At block 612, the reader device may be calibrated. In some embodiments, the reader device may be provided with one or more calibration algorithms to calculate one or more correction factors based on a blood-based reference measurement, analyte sensor configuration (e.g., diffusion characteristics of analyte sensor in interstitial fluid), optical correction parameters (e.g. skin characteristics at implantation site, analyte sensor implantation depth), relationship of target analyte concentration in blood to target analyte concentration in interstitial fluid (e.g., a predetermined blood-interstitial fluid conversion factor), previous analyte sensor readings/data trends, and/or other parameters. FIGS. 7a-n illustrate examples of user interface displays of a reader device 700 corresponding to various calibration operations.

Calibration of the reader device may be initiated by the user in some embodiments. For example, as illustrated in FIG. 7a, the reader device may provide a user interface with a display field 701 and an interactive menu 709 with user-selectable options for various operations related to mobile analyte monitoring. Interactive menu 709 may include an analyte reading option 703, calibration option 705, and review/export option 707 (e.g., physical or touchscreen buttons, links, etc.). If present, display field 701 may show instructions, a recent analyte concentration, or other relevant information. In other embodiments, reader device calibration may be initiated by the reader device and/or by a third party computer system based on a pre-determined schedule and/or prior analyte sensor readings.

In some embodiments, at block 612 the reader device may be provided with a reference measurement (e.g., a blood analyte concentration) obtained by another method (e.g., a blood test). For example, the reference measurement may be a blood glucose concentration obtained with a standard glucose test strip, a commercially available blood glucose meter, or other commercially available blood test/meter. Such measuring devices/systems are referred to herein as “reference testing devices.” The reader device may receive the reference measurement in various forms, including but not limited to text input, voice/auditory input from the user or a reference testing device that provides an auditory output, electronic signals from the reference testing, and/or optical input (e.g., a captured image of the reference testing device display showing the reference measurement). FIG. 7b illustrates an example of a user interface display with an interactive menu 719 that includes user-selectable options including electronic input option 711, optical input option 713, manual input option 715, and voice/auditory input option 717. Optionally, display field 701 may prompt the user to select one of the available input options. In some embodiments, the reader device may record/store an input in association with the time at which the input was received and/or other data specified by the user (e.g., description of a food or beverage consumed, medication taken, exercise, etc.).

FIGS. 7c and 7d illustrate examples of user interface displays provided by a reader device for electronic input of the reference measurement (e.g., in response to selection of electronic input option 711), in accordance with various embodiments. As shown for example in FIG. 7c, the reader device may provide one or more user-selectable options to allow the user to identify the type of reference testing device and/or electrical connection that will be used to provide the reference measurement.

FIGS. 7e-i illustrate examples of user interface displays provided by a reader device for optical input of the reference measurement (e.g., in response to selection of optical input option 713), in accordance with various embodiments. As shown for example in FIG. 7e, the reader device may activate the optical sensor (e.g., optical sensor 472) in preparation for capturing an image of the reference testing device. In some embodiments, the optical sensor may be a camera of a smartphone. The reader device may prompt the user to adjust one or more image capture parameters, such as the distance of the optical sensor from the reference testing device, lighting conditions, addition of a filter/lens, and/or the angle at which the optical sensor is positioned relative to the reference testing device (FIG. 7f). In some embodiments, the reader device may automatically capture the image upon determining that the image capture parameters are within acceptable ranges. For example, the reader device and/or optical sensor may include an autofocus function that focuses the optical sensor for image capture. In other embodiments, the image capture may be initiated by the user (e.g., by user operation of a button or other control feature).

Optionally, the reader device may provide an indication to the user that the image was successfully captured (FIG. 7g). In some embodiments, the reader device and/or a third party computer system may analyze the captured image to determine whether it is suitable for use to determine the reference measurement. If the captured image is determined to be suitable for use to determine the reference measurement, the reader device may provide an indication to the user that the image was successfully captured (FIG. 7h). If the captured image is determined to be unsuitable for use to determine the reference measurement (e.g., is out of focus or unreadable), the reader device may prompt the user to capture another image of the reference testing device (FIG. 7i).

FIGS. 7j-k illustrate examples of user interface displays provided by a reader device for auditory input of the reference measurement (e.g., in response to selection of voice/auditory input option 717), in accordance with various embodiments. As shown for example in FIG. 7j, the reader device may prompt the user to perform an action, such as activating a user-selectable control (e.g., tapping a touchscreen button, activating a physical key or button, etc.), in order to record the user's voice or auditory output of the reference testing device. Optionally, the reader device may prompt the user to perform another action (e.g., tapping a touchscreen button or activating another reader device control) to stop the recording.

FIG. 7l illustrates an example of a user interface display provided by a reader device for manual input of the reference measurement (e.g., in response to selection of manual input option 715), in accordance with various embodiments. In some embodiments, the reader device may prompt the user to enter the reference measurement as text input by activating physical or virtual keys, buttons, or other user-selectable features that represent alphanumeric characters. Optionally, the reader device may prompt the user to perform another action (e.g., tapping a touchscreen button or activating another reader device control) to confirm and/or save the manual input.

FIG. 7m illustrates an example of a user interface display provided by a reader device for confirmation of the reference measurement input, in accordance with various embodiments. In some embodiments, the reader device may display the reference measurement input (e.g., in display field 701). In other embodiments, the reader device may optionally provide an auditory indication of the reference measurement input (e.g., by generating/playing a recorded or simulated vocalization of the reference measurement input). Optionally, the reader device may prompt the user to confirm and/or save the manual input.

In various embodiments, one or more of the user interface displays may include additional user-selectable features (e.g., virtual buttons or keys, links, etc.) configured to provide control over, or access to, various options/displays of the imaging application. For example, some of the user interface displays may include an analyte reading option 703 that can be selected to initiate an analyte sensor reading process, as described further herein. In some embodiments, one or more of the user interface displays may include user-selectable features for accessing a user interface display for inputting calibration data, analyte sensor data, and/or other related data (e.g., add readings option 723), for accessing saved or tracked data such as calibration/analyte sensor data (e.g., view data option 725), for initiating a calibration process (e.g., calibration option 727, for accessing and/or adjusting one or more settings of the reader device and/or imaging application (e.g., settings option 727), and/or other options.

FIG. 7n illustrates an example of a user interface display that shows saved and/or tracked calibration data, in accordance with various embodiments. Optionally, such a user interface display may be provided in response to selection by the user of view data option 725. In some embodiments, a user interface display may include a user dashboard or menu 739 with relevant data arranged in any suitable manner for viewing by the user (e.g., arranged in columns, a table, a grid, etc.). For example, the user interface display may include a first field 731 that indicates a general time of day relative to a predetermined event (e.g., a specified meal such as breakfast, lunch, or dinner, sleep, waking, exercise, medication, consumption of carbohydrates, etc.). A time or date at which a reference measurement input was received by the reader device may be displayed in another field 733 relative to the corresponding reference measurement (field 735). Optionally, the user interface display may also show the input type or method for each of the reference measurements (field 737).

The reader device may be calibrated based at least in part on the reference measurement(s). The calibration process may be performed by the reader device, a third party computing system, and/or both. In some embodiments, the reader device and/or third party computing system may track reference measurement inputs as part of the calibration process. As illustrated for example in FIG. 7n, the reader device may track reference measurement inputs and/or associated data over a period of days, weeks, months, or years. In some embodiments, the reader device may periodically transmit the reference measurement inputs and/or associated data to a third party computing device. This may allow the reader device to store a smaller volume of tracking data in local storage. In some embodiments, tracking data may be accessed/downloaded by the reader device from the third party computing system (e.g., analyte sensor manufacturer, cloud network, etc.) in response to a request from the user for such data.

Optionally, at block 614, the reader device and/or third party computer system may update one or more calibration/image analysis algorithms. In some embodiments, a third party computer system may update a calibration/image analysis algorithm based at least in part on reference measurements and/or prior analyte sensor readings. For example, the third party computing system may periodically supply new algorithms to the reader device or update existing algorithms on the reader device, network/cloud, server, or other remote source based on one or more factors such as analyte sensor performance/stability data, analyte sensor lot performance/stability data, clinical trial data, and the like. In some embodiments, calibration/image analysis operations may be performed by a third party computing system, and block 614 may be omitted.

At block 616, the optical sensor of the reader device may be used to capture an image of the implanted analyte sensor. In some embodiments, the reader device may prompt the user to capture an image of the implanted analyte sensor within a particular amount of time relative to an event (e.g., before or after implantation of the sensor, obtaining a reference measurement with a reference testing device, consuming a quantity of a food or beverage). In other embodiments, a third party computer system or the reader device may prompt the user to capture an image of the implanted analyte sensor within a predetermined time period based on performance/stability data or tracked data (e.g., corresponding to the analyte sensor or other analyte sensors of the same manufacturing lot). In still other embodiments, the reader device may prompt the user to capture the image in accordance with reminders set by the user.

FIGS. 7o-s illustrate examples of user interface displays provided by a reader device for analyte sensor image capture, in accordance with various embodiments. As shown for example in FIG. 70, the reader device may activate the optical sensor (e.g., optical sensor 472) in preparation for capturing an image of the analyte sensor. In some embodiments, as shown for example in FIG. 7p, the reader device may prompt the user to adjust one or more image capture parameters, such as the distance of the optical sensor from the reference testing device, lighting conditions, and/or the angle at which the optical sensor is positioned relative to the reference testing device. Optionally, an image of the analyte sensor may be captured after implantation of the analyte sensor into the dermis of the user. FIG. 7p illustrates an image of the implanted analyte sensor 751 in the dermis of a user's arm.

In some embodiments, the reader device may automatically capture the image upon determining that the image capture parameters are within acceptable ranges. For example, the reader device and/or optical sensor may include an autofocus function that focuses the optical sensor for image capture. In other embodiments, the image capture may be initiated by the user (e.g., by user operation of a physical/virtual button or other control feature).

Optionally, the reader device may provide an indication to the user that the image was successfully captured (FIG. 7q). In some embodiments, the reader device and/or a third party computer system may analyze the captured image to determine whether it is suitable for use to determine the reference measurement. If the captured image is determined to be suitable for use to determine the reference measurement, the reader device may provide an indication to the user that the image capture was successful (FIG. 7r). If the captured image is determined to be unsuitable for use to determine the reference measurement (e.g., is out of focus or unreadable), the reader device may prompt the user to capture another image of the reference testing device (FIG. 7s).

At block 618, the captured image may be analyzed by the reader device and/or a third party computing system. In some embodiments, the image analysis may include one or more operations described herein with reference to FIG. 5.

At block 622, the captured image may be converted to an analyte concentration (e.g., a blood analyte concentration or an ISF analyte concentration) by the reader device and/or a third party computing system. In some embodiments, the reader device may perform the conversion. Alternatively, in other embodiments, the reader device may transmit the image data to a third party computer system at block 634. The third party computer system may generate and send instructions for converting the image data to the reader device. At block 636, the reader device may receive the instructions, and may convert the image data to an analyte concentration at block 622 based at least on the received instructions. In various embodiments, the conversion of image data to an analyte concentration may include one or more operations described herein with reference to FIG. 5.

At block 624, the determined analyte concentration may be communicated to the reader device (e.g., by a third party computer system performing the conversion) and/or to the user (e.g., by the reader device). For example, as shown in FIG. 7t, the analyte concentration may be displayed visually (e.g., in data field 701). Optionally, the analyte concentration may be provided as an auditory output.

At block 626, the reader device may receive data input from the user. In various embodiments, the user may input data such as foods/beverages consumed, time of consumption, calories consumed, blood glucose levels, physical activities, calibration data, insulin or other medication taken, time at which the medication was taken, quantity/dose of medication, weight, blood pressure, pulse rate, and/or various other data that may be relevant to the user's health and/or target analyte concentration. In some embodiments, the data input may include images of food, beverages, and/or medication captured by the reader device. The images may be time-stamped by the reader device, and related data input by the user at a later time (e.g., identification of the food/beverage/medication, quantity consumed, etc.) may be associated with the corresponding images/times. This may allow the user to enter such data into the reader device at a more convenient time while accurately recording the time at which the food/beverage/medication was consumed. Optionally, in some embodiments the reader device and/or third party computer system may analyze the captured image of the food/beverage/medications to determine additional data, such as nutritional content (e.g., based on an identifying characteristic such as a logo, symbol, text, or bar code on a wrapper, vial, container, etc.).

At block 628, the reader device may report one or more data trends to the user. For example, the reader device may report data trends to the user as a function of time (e.g., over a day, week, month, year, etc.) in the form of a dashboard, chart, table, or other format. FIG. 7u illustrates an example of a user interface display provided by the reader device at a corresponding stage of operation. As shown, the reader device may provide one or more user-selectable features that allow the user to view analyte concentrations 759 as a function of time (e.g., relative to mealtimes 755 and/or times 757 at which each analyte sensor reading was taken). The reader device may provide user-selectable options for viewing the readings over the course of a day (daily option 761), a week (weekly option 763), and/or a month (monthly option 765).

At block 630, the reader device may track one or more analyte sensor readings (e.g., analyte concentrations, image data, etc.) for a predetermined period of time. In some embodiments, the reader device may track analyte sensor readings, calibration data, and/or additional data input by the user for a period of seven days. In other embodiments, the reader device may track one or more such parameters for two weeks, a month, or another period of time.

Optionally, at block 632, the reader device may report tracked readings, data trends, and/or other relevant data (e.g., image data, conversion data, user inputs, etc.) to a third party computing system. In some embodiments, the reporting may be contingent upon approval by the user, and/or initiated by the user. In other embodiments, the reporting may be automatic and/or may not require the approval of the user. In still other embodiments, the reader device may report some types or categories of data (e.g., data trends, image data, conversion data) without user approval, but may report other types or categories of data (e.g., user inputs regarding meals, calories, weight, etc.) only upon receiving approval from the user. Optionally, the third party computing system may be, or may include, a computing system of the analyte sensor manufacturer and/or a developer of the imaging application and/or algorithm(s) thereof.

At block 638, the third party computing system may analyze the reported data to assess performance and/or stability characteristics of the analyte sensor. In some embodiments, the third party computing system may be a computing system of the analyte sensor manufacturer. Received data may be analyzed to assess the performance and/or stability of individual analyte sensors and/or analyte sensor lots. For example, as described with reference to FIG. 5, image data corresponding to a particular analyte sensor may be analyzed to determine whether the analyte sensor is malfunctioning, leaking, or exhibiting responses outside of an expected range of response. As another example, the reported data may include analyte sensor insertion/positioning data (e.g., image data), and the third party computing system may assess the position of the analyte sensor within the dermis. The third party computing system may use the assessment to identify stability or performance issues resulting from, or associated with, incorrect analyte sensor insertion/placement, and to distinguish such issues from stability/performance issues caused by other factors.

In some embodiments, the third party computing system may perform further operations in response to analysis of the reported data for the analyte sensor and/or analyte sensor lot. In various examples, the third party computing system may: update one or more algorithms; recommend changes to the frequency or timing of recalibrations/analyte sensor readings; determine and communicate to the reader device or user a predicted useful life of the analyte sensor; determine that the analyte sensor is malfunctioning or should be replaced (e.g., based on variance between expected readings or values and actual readings or values, variance between an expected response pattern and an actual response pattern, change in skin optical characteristics, variance between duplicate analysis regions, etc.); generate recommendations for adjustments to dose/timing of medication based on data trends (e.g., taking insulin 25 minutes prior to a meal instead of 30 minutes before a meal, increasing or decreasing the dosage, calibration of oral mediation, efficacy of medication, predicted increase in efficacy if adjusted as recommended); generate other recommendations for the user based on user data (e.g., alert user that self-reported carbohydrate consumption underestimates or overestimates actual carbohydrate consumption); and/or generate other recommendations for the user based on group or analyte sensor lot data (e.g., recommend a change in frequency or timing of recalibration/analyte sensor readings based on a determination that performance/stability was increased in other analyte sensors as a result of the same or similar change).

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. An analyte monitoring system, comprising:
an analyte sensor having a base and an analyte reagent system coupled to the base, wherein the analyte sensor is configured to exhibit a reversible color change in response to a target analyte; and
a reader device configured to capture an image of the analyte sensor and determine a concentration of the target analyte based at least on the image.

2. The analyte monitoring system of claim 1, wherein the reader device is a mobile electronic device selected from the group consisting of a cell phone, a smart phone, a personal computer, and a personal digital assistant.

3. The analyte monitoring system of claim 1, wherein the base comprises a polymeric material impregnated with TiO₂.

4. The analyte monitoring system of claim 1, wherein the base is reflective and comprises a metal.

5. The analyte monitoring system of claim 1, wherein the analyte sensor is configured to be inserted into an animal.

6. The analyte monitoring system of claim 1, wherein the analyte sensor is configured to be inserted into the dermis of the animal.

7. The analyte monitoring system of claim 1, further comprising a medical device communicatively coupled to the reader device.

8. The analyte monitoring system of claim 7, wherein the medical device is an insulin pump.

9. The analyte monitoring system of claim 1, wherein the analyte reagent system comprises one or more of a lipophilic anion, a chromoionophore, and an ionophore.

10. The analyte monitoring system of claim 1, wherein the analyte reagent system comprises an enzyme.

11. The analyte monitoring system of claim 1, wherein the analyte sensor has a total thickness of 50 μm or less.

12. The analyte monitoring system of claim 9, wherein the enzyme is glucose oxidase.

13. The analyte monitoring system of claim 1, wherein the image comprises a representation of at least one analyte measurement chamber.

14. The analyte monitoring system of claim 1, wherein the analyte sensor is configured to provide a qualitative indication of analyte concentration that is visible to the user.

15. The analyte monitoring system of claim 6, wherein the animal is a human, the reader device comprises an image capture device and a smart phone in wireless communication with the image capture device, and the image capture device is configured to be retained on the animal's body proximal to the analyte sensor.

16. A method of monitoring an analyte sensor implanted in the dermis of a subject, wherein the implanted analyte sensor includes one or more analysis regions configured to exhibit a reversible color change in response to a change in concentration of a target analyte, the computer-implemented method comprising:
receiving first image data representative of a first image of the implanted analyte sensor;
determining, based at least on the first image data, a first color value corresponding to a portion of the one or more analysis regions; and
determining a concentration of the target analyte based at least on the first color value.

17. The method of claim 16, wherein the first image of the implanted analyte sensor is captured by an optical sensor of a reader device.

18. The method of claim 17, wherein the reader device is a smart phone.

19. The method of claim 17, wherein said determining the concentration of the target analyte is performed by a third party computer system, the method further comprising communicating the concentration of the target analyte to the reader device.

20. The method of claim 17, further comprising:
receiving second image data representative of a second image of the implanted analyte sensor;
determining, based at least on the second image data, a second color value corresponding to a portion of the implanted analyte sensor;
determining a difference between the first color value and the second color value; and
determining, based at least on said difference, one or more of a correction factor, a sensor malfunction, and a time frame for replacement of the implanted analyte sensor.

21. The method of claim 17, wherein said determining the concentration of the target analyte is performed by the reader device, the method further comprising communicating the first image data, the first color value, or the concentration of the target analyte to a computing system of a manufacturer of the implanted analyte sensor.

22. The method of claim 17, wherein said determining one or more of the correction factor, the sensor malfunction, and the time frame for replacement of the implanted analyte sensor is performed by the computing system of the manufacturer of the sensor.

23. The method of claim 17, wherein the reader device includes an image capture device and a smart phone in wireless communication with the image capture device, the image capture device configured to be retained on the subject's body proximal to the implanted analyte sensor.

24. The method of claim 23, wherein the image capture device is configured to capture images at predetermined intervals or in response to a command from the reader device.

25. The method of claim 19, further including:
receiving by the third party computer system, from the reader device, user input data regarding one or more meals or a medication; and
generating by the third party computer system, based at least on the user input data, one or more recommendations to the user regarding said medication.

26. A non-transitory computer readable medium comprising instructions operable, upon execution by a processor of an electronic device, to cause the electronic device to:
receive a first image of an analyte sensor implanted in the dermis of a subject, wherein the implanted analyte sensor includes one or more analysis regions configured to exhibit a reversible color change in response to a change in concentration of a target analyte;
determine, based at least on the first image, a first color value corresponding to a portion of the one or more analysis regions; and
determine a concentration of the target analyte based at least on the first color value.

27. The non-transitory computer readable medium of claim 26, wherein the electronic device is a smart phone.

28. The non-transitory computer readable medium of claim 26, wherein the electronic device is a third party computer system and the first image of the implanted analyte sensor is captured by an optical sensor of a reader device.

29. The non-transitory computer readable medium of claim 28, wherein the reader device is a smart phone.

30. The non-transitory computer readable medium of claim 26, wherein the instructions are further operable, upon execution by the processor, to cause the electronic device to:
determine, based at least on the first image, a second color value corresponding to a second portion of the implanted analyte sensor;
determine a difference between the first color value and the second color value; and
determine, based at least on said difference, one or more of a correction factor, a sensor malfunction, and a time frame for replacement of the implanted analyte sensor.

31. The non-transitory computer readable medium of claim 26, wherein the instructions are further operable, upon execution by upon execution by the processor, to cause the electronic device to communicate one or more of the first image and the first color value to a third party computer system.

32. The non-transitory computer readable medium of claim 26, wherein the instructions are further operable, upon execution by the processor, to cause the electronic device to:
receive user input data regarding one or more meals or a medication; and
based at least on the user input data, generate one or more recommendations to the user regarding said medication.

33. The non-transitory computer readable medium of claim 17, wherein the instructions are further operable, upon execution by the processor, to cause the electronic device to:
capture a plurality of images of the analyte sensor; and
automatically adjust one or more image capture parameters based on one or more of the images.

34. The non-transitory computer readable medium of claim 33, wherein the plurality of images are captured as a video, and the image capture parameters are adjusted during capture of the video.
